(19) United States
(12) Patent Application Publication
(10) Pub. No.: US 2025/0281954 A1
(43) Pub. Date: Sep. 11, 2025

(54) CLOSED-LOOP TREATMENT OF CONTAMINATED SOIL

(71) Applicant: Remedy Scientific Inc., Piedmont, CA (US)

(72) Inventors: Randol W. AIKIN, Piedmont, CA (US); Jorah R. WYER, Boise, ID (US); Keith A. HALL, San Jose, CA (US); Ravi SRINIVASAN, Saratoga, CA (US); Edmund H. ANTELL, Oakland, CA (US); Darren L. KRAABEL, Monteagle, TN (US)

(21) Appl. No.: 19/071,674

(22) Filed: Mar. 5, 2025

Related U.S. Application Data
(60) Provisional application No. 63/562,223, filed on Mar. 6, 2024.

(51) Int. Cl. B09C 1/08; G06F 30/20

(52) U.S. Cl. CPC B09C 1/08; G06F 30/20

(57) ABSTRACT

A system for on-site treatment of impacted soil may include a soil-treatment device, a sensing device, and a computing device. The soil-treatment device includes an inlet for receiving impacted soil from a contaminated site and a reactor configured to treat the impacted soil according to one or more operational parameters. The sensing device is configured to receive a sample from the impacted soil and perform, on-site during the treatment process, a measurement of the sample. The computing device includes a memory storing executable instructions and one or more processors. When executed, the instructions cause the processors to receive the measurement from the sensing device, apply a model to determine, during the treatment process and based on the measurement, a value of one of the operational parameters in the set, and output the determined value for the soil-treatment device to perform the treatment operation according to the operational parameters.

### CROSS REFERENCE TO RELATED APPLICATION(S)

This application claims the benefit of U.S. Provisional Application No. 63/562,223, filed on Mar. 6, 2024, which is incorporated by reference herein in its entirety for all purposes.

### BACKGROUND

In conventional soil remediation methods, treatment processes are typically designed based on static or pre-determined contamination levels without real-time adaptability. One common approach involves manually collecting soil samples from various points at a contaminated site and determining the highest detected contamination level. However, this practice often results in an overestimation of contamination, leading to unnecessary resource consumption and excessive treatment costs. The entire site is often treated as if it contains the highest measured contamination level, even though the actual contamination varies significantly within different soil batches. This results in inefficiencies where a large portion of the soil is over-treated, increasing operational costs and energy consumption.

Thermal desorption has also been widely used for soil remediation, particularly for PFAS contamination. This process involves heating the contaminated soil to high temperatures to volatilize and break down contaminants. However, thermal treatments are energy-intensive, requiring significant power input, and may pose the risk of releasing airborne pollutants. Moreover, thermal desorption does not always fully degrade PFAS compounds, potentially leading to incomplete remediation.

Thus, existing remediation technologies suffer from high operational costs, excessive energy consumption, and inefficient contaminant degradation.

### SUMMARY

In some embodiments, the disclosure described herein relate to a soil treatment system that includes a soil-treatment device. The soil-treatment device may include an inlet for receiving impacted soil from a contaminated site, and a reactor configured to carry out a treatment operation to treat the impacted soil according to a set of one or more operational parameters. The system may also include a sensing device that is on site with the soil treatment device. The sensing device is configured to receive a sample from the impacted soil, and perform, on site during a treatment process of impacted soil, a measurement of the sample. The soil treatment system may also include a computing device including memory and one or more processors. The memory stores executable instructions. The executable instructions, when executed by the one or more processors, cause the one or more processors to: receive the measurement of the sample performed by the sensing device; apply a model to determine, during the treatment process of the impacted soil and based on the measurement of the sample, a value of one of the operational parameters in the set; and output the value for the soil treatment device to carry out the treatment operation according to the set of one or more operational parameters.

In some embodiments, the reactor includes a ball mill reactor that includes a rotary shaft and metallic ball bearings, the treatment operation includes stirring the impacted soil with the metallic ball bearings at a rotational speed, and the rotational speed is adjustable and is one of the operational parameters in the set.

In some embodiments, the reactor is configured to operate at a temperature that is below 50 degrees Celsius and below 1.5 times atmospheric pressure.

In some embodiments, the reactor is configured to receive aluminum and magnesium metallic additives for the treatment operation, and an amount of the metallic additive is one of the operational parameters.

In some embodiments, the sensing device is an automatic sensor that is installed within the soil-treatment device.

In some embodiments, the sensing device include one or more of: a direct ionization mass spectrometer, a near-infrared (NIR) spectroscopy sensor, thermal sensor, or optical and spectral sensors.

In some embodiments, the sensing device is a measurement device separated from the soil-treatment device, and the sensing device includes laboratory kits to measure the sample during the treatment process.

In some embodiments, the treatment process includes a pre-operation measurement and the treatment operation.

In some embodiments, the set of one or more operational parameters includes one or more of: a dwell time, a milling speed, a chemical additive amount, or an energy input.

In some embodiments, the model is a generalized additive model and the generalized additive model is configured to predict adjustments to operational parameters based on one or more sensed soil parameters.

In some embodiments, the model is a convolutional neural network and the convolutional neural network is configured to analyze hyperspectral imaging data from Near-Infrared (NIR) spectroscopy to determine hidden features in soil composition and contamination patterns.

In some embodiments, the measurement performed by the sensing device includes one or more of: a contaminant concentration, a moisture content, an organic carbon measurement, and a mineral composition.

In some embodiments, the model includes algorithms for: measuring soil parameters; estimating operational parameters based on the soil parameters; receiving energy cost data; and adjusting the operational parameters based on the energy cost data.

In some embodiments, the executable instructions, when executed, further cause the one or more processors to: determine an estimated operational parameter based on soil parameters; calculate the estimated destruction rate; identify a target treatment end point; provide the estimated destruction rate, the target treatment end point, and the estimated operational parameter into an optimization algorithm to adjust the operational parameter based on cost; and output a recommended action that includes one or more of: a pre-treatment step, a post-treatment step, or a value of the operational parameter.

In some embodiments, the impacted soil is a first batch of impacted soil from a contaminated site, the contaminated site includes the first batch and a second batch of impacted soil, and wherein the second batch of impacted soil has a level of contamination that is at least 10 times different from the first batch, and the computing device is configured to determine batch-specific operational parameters for treating the batches.

In some embodiments, the model is configured to determine the value of one of the operational parameters based on energy cost.

In some embodiments, the disclosure described herein relate to a method for treating soil with PFAS contamination. The method may include: causing an excavation of impacted soil from a contaminated site; performing, on site during a treatment process of the impacted soil, a measurement of the sample; applying a model to determine, during the treatment process of the impacted soil and based on the measurement of the sample, a value of an operational parameter for operating a soil-treatment device that carries out the treatment process that includes a treatment to treat the impacted soil according to the operational parameter; and updating the operational parameter during the treatment process based on live data of further measurements of samples from the impacted soil.

### BRIEF DESCRIPTION OF THE DRAWINGS

FIG. 1 is a block diagram of an example soil treatment system environment, in accordance with some embodiments.

FIG. 2 is a perspective view of a treatment device, in accordance with some embodiments.

FIG. 3 is a perspective view of an example ball milling chamber is illustrated, in accordance with some embodiments.

FIG. 4 is a flowchart depicting a soil treatment process, in accordance with some embodiments.

FIG. 5 is a block diagram depicting a data processing pipeline, in accordance with some embodiments.

FIG. 6 is a conceptual diagram illustrating an example algorithm that is modeled after a “Sense,” “Plan,” “Act” strategy, in accordance with some embodiments.

FIG. 7 is a flowchart depicting an example of an optimization algorithm, in accordance with some embodiments.

FIG. 8 is a block diagram illustrating an example neural network, in accordance with some embodiments.

FIG. 9 is a block diagram illustrating components of an example computing machine, in accordance with some embodiments.

The figures depict, and the detailed description describes, various non-limiting embodiments for purposes of illustration only.

### DETAILED DESCRIPTION

The figures (FIGs.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

The disclosed system provides a closed-loop solution for on-site soil remediation, dynamically optimizing treatment conditions in real time. The system includes a soil-treatment device that receives impacted soil and applies a treatment operation to degrade contaminants. A sensing device performs in-process measurements of soil properties, including concentration, moisture content, and organic carbon composition. A computing device processes the measurement data, applies a predictive model, and determines optimal operational parameters such as milling speed, chemical additive dosage, and dwell time. The computing device continuously updates these parameters throughout the treatment process based on live sensor feedback, ensuring efficient contaminant degradation while minimizing energy consumption and additive use. The predictive model may incorporate rule-based logic, heuristic estimations, and machine-learning algorithms, including Generalized Additive Models (GAMs) and Convolutional Neural Networks (CNNs), to refine operational parameters dynamically. By integrating real-time sensing with machine-learning-driven process control, the system enables adaptive soil treatment, improving throughput, reducing resource waste, and ensuring regulatory compliance. This closed-loop automation enhances the efficiency of soil remediation by adjusting treatment conditions on a batch-specific basis, allowing for optimized energy and cost management across varying contamination levels.

### System Overview

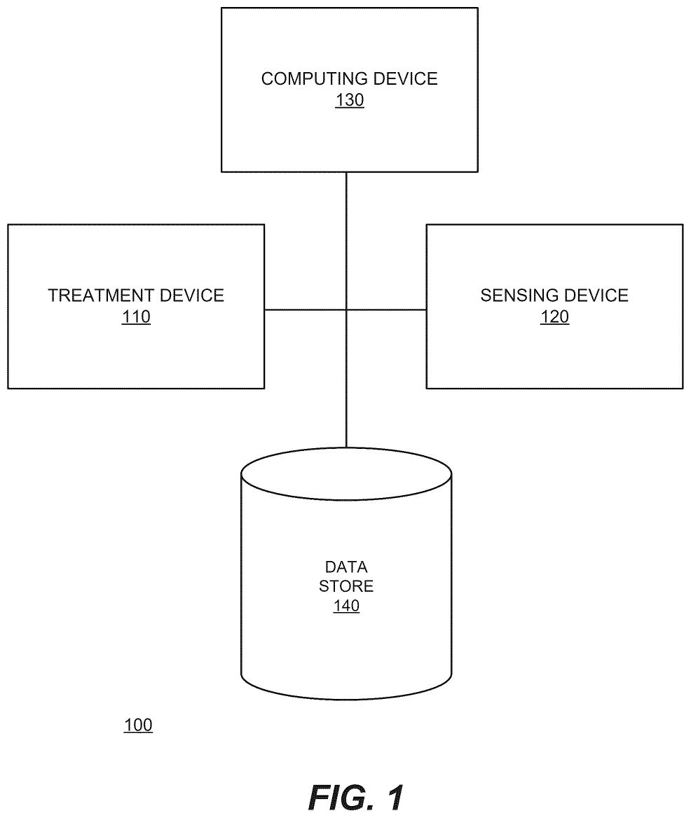

Referring now to Figure FIG. 1, shown is a block diagram illustrating an embodiment of an example soil-treatment system environment 100 for carrying out an on-site soil treatment process, in accordance with some embodiments. By way of example, the system environment 100 includes a treatment device 110, a sensing device 120, a computing device 130, and a data store 140. The entities and components in the system environment 100 may communicate with each other through a network or locally. In various embodiments, the system environment 100 may include fewer or additional components. The system environment 100 also may include different components.

The components in the system environment 100 may each correspond to a separate and independent entity or may be controlled by the same entity. For example, in some embodiments, the treatment device 110 and the computing device 130 are operated by the same entity. In some embodiments, the computing device 130 and a data store 140 can be operated by different entities, such as in situations where the data store 140 is operated by a third-party Cloud service provider.

The components in the soil-treatment system environment 100 may be geographically located in the same location or distributed in various locations. In some embodiments, the components in treatment system 110 may be geographically located in the same physical housing with a sensing device 120, a computing device 130, and a data store 140, consolidating the functionalities into a single integrated soil-treatment system, providing a smart and integrated closed-loop system. In some embodiments, the components can be distributed across various devices, offering flexibility and scalability. For instance, the sensing device 120 can be a device that is commercially available from a third party, while the computing device 130 is a separate computer, such as a desktop computer located at the treatment site for controlling one or multiple treatment devices 110. In some embodiments, the computing device 130 and data store 140 may also take the form of a remote server, such as a computing server serving as the core of a Software as a Service (SaaS) platform that provide automated control and analytics of remotes treatment systems 110. This remote server may serve as a centralized control for a variety of treatment devices 110 in different soil treatment sites.

While each of the components in the system environment 100 is sometimes described in disclosure in a singular form, the system environment 100 may include one or more of each of the components. For example, different types of sensing device 120 may be used to various parameters or measurements of soil samples from a treatment device 110. Likewise, while on some occasions a component may be described in a plural form, in various embodiments, the component may be present in a single instance.

In some embodiments, a treatment device 110 may be a physical apparatus that facilitates soil remediation by breaking down contaminants through mechanical and/or chemical processes. The treatment device 110 may include a reactor designed to optimize the degradation of contaminants in soil batches. For example, the treatment device 110 may incorporate a ball milling system where mechanical agitation and chemical additives interact with soil particles to accelerate contaminant breakdown. The treatment device 110 may include a rotating chamber that operates at variable speeds to enhance reaction efficiency. In some embodiments, the treatment device 110 may further include a thermally controlled shell to maintain reaction stability, a robotic material handling system for automated batch processing, and an integrated sampling mechanism to extract soil samples for real-time contamination analysis. These are merely example components of a treatment device 110 and the precise arrangement and components of the treatment device 110 may vary, depending on the treatment sites, types of treatment techniques, and target contaminants.

A treatment device 110 may be specialized one or may be a multipurpose one that is configured to handle a wider range of contaminants. Examples of soil contaminants may include perfluoroalkyl and polyfluoroalkyl substances (PFAS), heavy metals, chlorinated solvents, petroleum hydrocarbons, polychlorinated biphenyls (PCBs), pesticides, dioxins,, volatile organic compounds (VOCs), semi-volatile organic compounds (SVOCs), and cyanides. A specialized treatment device 110 may be designed with physical components that are specific and effective against a particular type of soil contaminant. Some physical components in a specialized treatment device 110 may be known in the art but conventionally are not optimized based on batch-specific information.

In some embodiments, in treating PFAS, a treatment device 110 may include a ball milling system configured to facilitate the breakdown of contaminants in soil through mechanical energy and chemical additives. For example, the treatment device 110 may include a high-energy ball mill that includes a chamber containing a central agitator and milling media, such as steel or ceramic ball bearings. The chamber and the central agitator may be rotating relative to each other, whether one component is being stationary. The treatment device 110 may subject impacted soil to high-impact collisions between the milling media and the soil particles, promoting particle size reduction and the generation of localized high-energy conditions that facilitate contaminant degradation. In some embodiments, the treatment device 110 may operate at variable rotational speeds, allowing for adjustments to milling intensity based on the contamination level of the soil batch. The treatment device 110 may be configured to handle PFAS as well as other contaminants, such as chlorinated solvents, and petroleum hydrocarbons.

In some embodiments, a treatment device 110 may include chemical additive dosing to enhance the degradation of PFAS during ball milling. In some embodiments, the treatment device 110 may introduce metal-based additives, such as aluminum or magnesium, which react with PFAS molecules under high-energy mechanical conditions to promote defluorination and oxidation reactions. In some embodiments, the treatment device 110 may include a real-time sensing system that measures contaminant degradation progress during milling and adjusts milling parameters accordingly. The treatment device 110 may operate at energy densities that exceed conventional ball mills used in mining and materials processing, allowing for accelerated degradation kinetics of persistent contaminants. In some embodiments, the treatment device 110 may be configured to operate at ambient temperature and atmospheric pressure, preventing volatilization of hazardous compounds while achieving effective contaminant destruction.

While ball milling is used as an example for treating PFAS contamination, a treatment device 110 may also take different forms that do not use ball milling. A treatment device 110 configured to facilitate PFAS remediation may use various treatment methodologies. In some embodiments, the treatment device 110 may include a filtration-based system, such as a granular activated carbon (GAC) system, in which activated carbon is used to absorb PFAS from contaminated soil or water. In some embodiments, the treatment device 110 may incorporate an ion exchange resin system, where specialized resins selectively capture PFAS compounds from contaminated media. In some embodiments, a treatment device 110 may include a membrane-based separation system, such as a reverse osmosis system, where contaminated water is passed through a semi-permeable membrane to remove PFAS, generating a concentrated waste stream that may undergo further treatment. In some embodiments, the treatment device 110 may include an electrochemical oxidation system, in which an electric current is applied to generate reactive species that facilitate PFAS degradation. In some embodiments, the treatment device 110 may include a plasma-based reactor, where plasma-generated reactive species interact with PFAS molecules to promote contaminant breakdown. In some embodiments, the treatment device 110 may use a water oxidation system, in which PFAS-contaminated media is exposed to elevated temperature and pressure conditions to induce oxidative decomposition. In some embodiments, a treatment device 110 may include a thermal-based remediation system. In some embodiments, the treatment device 110 may be configured as a thermal desorption unit, where contaminated soil is heated to a temperature sufficient to volatilize PFAS, followed by secondary capture and treatment of the contaminants by ball milling or other devices. In some embodiments, the treatment device 110 may be an incineration system, in which PFAS-contaminated material is subjected to high-temperature combustion, typically exceeding 1,100° C., to facilitate complete thermal degradation of PFAS compounds. In some embodiments, a treatment device 110 may include a biological remediation system, such as a bioaugmentation or bioremediation system, where microorganisms are introduced to facilitate the breakdown of PFAS under controlled environmental conditions. In some embodiments, a treatment device 110 may include an in situ permeable reactive barrier (PRB), where a subsurface barrier containing reactive media, such as activated carbon or zero-valent iron, is installed to capture or degrade PFAS as groundwater flows through the treatment zone. In some embodiments, a treatment device 110 may integrate multiple remediation techniques to optimize contaminant degradation based on site-specific conditions and treatment objectives.

While treatment of PFAS is often described as primary examples in this disclosure, other types of treatment devices 110 may also be possible in various embodiments for other types of contaminants. For example, a treatment device 110 may be used to remediate petroleum hydrocarbons and fuel contaminants in soil using thermal and biological treatment methods. For example, the treatment device 110 may include a thermal desorption system, where contaminated soil is heated to volatilize hydrocarbons, which are then captured and treated using secondary combustion or condensation units. In some embodiments, the treatment device 110 may include an in-situ bioremediation system, where microbial consortia capable of degrading petroleum hydrocarbons are introduced into the contaminated soil, with treatment conditions such as oxygenation, nutrient supplementation, and moisture levels dynamically adjusted to optimize microbial degradation efficiency. In some embodiments, the treatment device 110 may incorporate a vapor extraction system that applies a vacuum to contaminated soil, facilitating the removal of volatile hydrocarbon contaminants, which may then be treated using activated carbon filtration or catalytic oxidation.

In some embodiments, a treatment device 110 may be configured to remediate heavy metal contamination in soil using chemical stabilization and electrokinetic treatment. In some embodiments, the treatment device 110 may include a chemical stabilization system, where soil is mixed with binding agents such as phosphate-based compounds, sulfides, or silicates, which react with heavy metals to form stable, insoluble complexes. In some embodiments, the treatment device 110 may include an electrokinetic remediation system, where an electric field is applied to contaminated soil, facilitating the migration of heavy metal ions toward electrode wells for subsequent excavation and treatment. In some embodiments, the treatment device 110 may incorporate a soil washing system, where contaminated soil is processed with chelating agents or surfactants to desorb heavy metals, followed by separation and immobilization of the excavated contaminants. In some embodiments, the treatment device 110 may include a phytoremediation system, where specific plant species are cultivated in contaminated soil to uptake and accumulate heavy metals, with harvested biomass subsequently processed to recover or safely dispose of the contaminants.

In some embodiments, treatment devices 110 may take various forms on-site remediation of contaminated soil. Some of the treatment device 110 may allow for in-situ treatment approaches while others may allow for ex-situ approaches. For ex-situ remediation, soil is excavated and treated in a controlled setting in a treatment device 110. For example, the treatment device 110 may process soil within a controlled reactor with adjustable parameters treatment parameters such as dwell time, chemical additive dosing, and energy input. In some embodiments, the treatment device 110 may be implemented as an in-situ treatment system, where contaminants are degraded or stabilized without the need for soil excavation. For example, chemical injection, electrokinetic treatment, or thermal methods may be applied directly to the impacted area. A treatment device 110 may be deployed directly into the impacted soil. In some embodiments, the treatment device 110 may include a modular system that can be positioned at a remediation site to minimize transportation costs and logistical complexities associated with off-site treatment.

In some embodiments, a treatment device 110 may be deployed at various contamination sites, including former industrial facilities, military installations, landfills, fuel storage depots, and chemical manufacturing plants. Contamination sites may also include areas impacted by historical waste disposal practices, such as decommissioned steel mills, refineries, mining operations, and sites affected by firefighting foam runoff, airports and military facilities. In some embodiments, impacted soil types may vary significantly based on the site's geological characteristics, including sandy soils, clay-rich soils, silty loams, peat, shale-derived soils, alluvial deposits, and gravelly substrates. The physical and chemical properties of the soil may influence the retention and mobility of contaminants. The treatment device 110 may carry out site-specific treatment adjustments to optimize remediation effectiveness.

In some embodiments, the degree of contamination within a site may vary significantly, even over short spatial distances, leading to heterogeneity in contaminant distribution. Contamination concentrations can range from trace levels to orders of magnitude higher in adjacent locations. The soil from a contaminated site, whether the soil has a measurable degree of contamination, may be referred to as impacted soil or contaminated soil. A treatment device 110 may be configured to account for this variability by dynamically adjusting treatment parameters based on real-time contamination analysis. In some embodiments, a sensing device 120 may detect contaminant levels in different soil batches. For example, at a PFAS-contaminated site, one soil sample may contain single digit parts-per-billion (PPB) levels of PFAS, while another sample from a nearby hot spot may contain concentrations hundreds of parts-per-million (PPM) levels that are from 10 times to ten of thousands times higher than other impacted soil samples from the same site.

In various embodiments, a treatment device 110 may take different configurations depending on the specific treatment methodology applied. In some embodiments, the treatment device 110 may include a batch-processing reactor where contaminated soil is treated in discrete quantities. In some embodiments, the treatment device 110 may be designed for continuous operation, where soil flows through the reactor at a controlled rate to optimize treatment throughput. The treatment device 110 may utilize different types of mechanical reactors, such as ball mills, stirred media mills, attrition mills, or high-shear reactors, to facilitate contaminant degradation. In some embodiments, the treatment device 110 may include an automated dosing system that introduces chemical additives based on real-time contamination analysis. The treatment device 110 may incorporate sensors for temperature monitoring, reaction energy control, and chemical concentration measurement to adjust treatment conditions dynamically. In some embodiments, the treatment device 110 may operate at varying energy densities, with energy input parameters determined based on the type and concentration of contaminants present in the soil batch. The treatment device 110 may be capable of treating multiple types of contaminants, such as halogenated organic compounds, pesticides, and industrial waste byproducts, using the same underlying mechanical treatment process.

In some embodiments, a treatment device 110 may function as part of a closed-loop remediation system that dynamically adjusts operational parameters based on real-time contamination data. The treatment device 110 may receive pre-treatment contamination analysis data from a sensing device 120, which may assess soil parameters such as contaminant concentration, moisture content, and organic carbon composition. The computing device 130 may analyze data from the sensing device 120 and determine improved operational parameters, including dwell time, milling speed, and additive concentrations. The treatment device 110 may then process the impacted soil under the selected operational parameters and extract post-treatment samples for contamination verification. In some embodiments, the treatment device 110 may be integrated with a real-time feedback loop where post-treatment contamination measurements inform further adjustments to the processing parameters.

In some embodiments, a sensing device 120 may be a device that is on site with the treatment device 110 and performs a measurement of a sample of impacted soil during a treatment process of impacted soil. The sensing device 120 may be configured to analyze soil properties, contamination levels, and process conditions in real time. The sensing device 120 may include various sensors, such as direct ionization mass spectrometers, near-infrared spectrometers, optical sensors, and thermal sensors. These sensors may detect contaminants, monitor soil composition, and assess parameters such as temperature, moisture, and mineral content. In some embodiments, the sensing device 120 may be integrated into an automated sample handling system to extract, analyze, and return soil samples for real-time feedback on treatment efficacy. The sensing device 120 may operate in conjunction with a computing device 130 to provide continuous monitoring and adjustment of treatment parameters.

In some embodiments, a treatment process may include multiple stages, including pre-processing, processing, and post-processing, each of which may be dynamically adjusted based on real-time data from a sensing device 120. The treatment process may begin with pre-processing, where soil samples are collected and characterized before treatment. The sensing device 120 may measure parameters such as moisture content, organic carbon levels, mineral composition, and contaminant concentration. These initial measurements may be used by a computing device 130 to determine the necessary adjustments to treatment parameters. In some embodiments, pre-processing may also include modifications to the soil, such as drying, particle size reduction, or pH adjustments, to improve treatment efficiency. The selection and duration of the pre-processing phase may vary depending on the complexity of the soil matrix and the specific treatment objectives.

In some embodiments, the processing phase may involve both real-time measurement and active treatment within a treatment device 110. The sensing device 120 may take an initial measurement of the soil within the treatment device 110 before activating the treatment cycle. The measurement phase may last from a few seconds to several minutes, depending on the complexity of the sample and the number of parameters analyzed. Following measurement, the treatment device 110 may apply mechanical, chemical, or thermal treatment to the soil to break down contaminants. The active treatment time may range from a few minutes to several hours, depending on the contaminant type, soil properties, and target remediation levels. In some embodiments, the treatment device 110 may be configured for continuous processing, where soil is gradually fed into the system and treated in real time. Alternatively, in some embodiments, the treatment device 110 may operate in a batch mode, where discrete soil samples undergo a defined treatment cycle before being analyzed again by the sensing device 120 to determine treatment effectiveness. The computing device 130 may dynamically adjust parameters such as dwell time, milling speed, chemical additive concentration, or energy input based on real-time feedback from the sensing device 120.

In some embodiments, post-processing may involve additional measurement and validation steps to confirm that treatment has achieved target contaminant reduction levels. The sensing device 120 may analyze treated soil to determine whether contaminants have been sufficiently degraded. If necessary, the computing device 130 may initiate further treatment cycles for soil that has not reached the desired endpoints. In some embodiments, post-processing may include stabilization of the treated soil, such as adjusting moisture levels or adding conditioning agents for site restoration. The treated soil may then be returned to the original excavation site, repurposed for other applications, or transported for further testing. The post-processing phase may also include data logging in a data store 140, where all treatment parameters, sensor readings, and contamination reduction data are recorded for regulatory compliance and process optimization. In some embodiments, the computing device 130 may use post-processing data to refine treatment algorithms, improving efficiency and cost-effectiveness for future remediation efforts.

In some embodiments, the processing phase may include both measurement and active treatment within a treatment device 110. Active treatment may or may not be down during the measurement. The sensing device 120 may take an initial measurement of the soil within the treatment device 110 before activating the treatment cycle. The measurement phase may last from a few seconds to several minutes, depending on the complexity of the sample and the number of parameters analyzed. Following measurement, the treatment device 110 may apply mechanical, chemical, or thermal treatment to the soil to break down contaminants. The active treatment time may range from a few minutes to several hours, or even days, depending on the contaminant type, soil properties, and target remediation levels. In some embodiments, the treatment device 110 may be configured for continuous processing, where soil is gradually fed into the system and treated in real time. Alternatively, or additionally, in some embodiments, the treatment device 110 may operate in a batch mode, where discrete soil samples undergo a defined treatment cycle before being analyzed again by the sensing device 120 to determine treatment effectiveness. The computing device 130 may dynamically adjust operational parameters such as dwell time, milling speed, chemical additive concentration, or energy input based on real-time feedback from the sensing device 120.

In some embodiments, post-processing may involve additional measurement and validation steps to confirm that treatment has achieved target contaminant reduction levels. The sensing device 120 may analyze treated soil to determine whether contaminants have been sufficiently degraded. Further treatment cycles may be performed for soil that has not reached the desired endpoints. In some embodiments, post-processing may include stabilization of the treated soil, such as adjusting moisture levels or adding conditioning agents for site restoration. The treated soil may then be returned to the original excavation site, repurposed for other applications, or transported for further testing. The post-processing phase may also include data logging in a data store 140, where all treatment parameters, sensor readings, and contamination reduction data are recorded for regulatory compliance and process optimization.

In some embodiments, sensing devices 120 may take different forms, such as sensors that are integrated within a treatment device 110, onsite analysis equipment for sample characterization, or a combination of both. For instance, in some embodiments, the sensing device 120 may take the form of an onboard sensor within the treatment device 110 that collects and analyzes data as soil undergoes treatment. In some embodiments, the sensing device 120 may be an independent analysis workstation that receives soil samples for detailed chemical and physical examination. For example, the sensing device 120 may include laboratory and analytical equipment used for analyzing various physical and/or chemical properties of soil samples during the treatment process, such as the level of contaminant. In some embodiments, the sensing device 120 may incorporate a distributed sensing network, with multiple sensing units deployed at various locations on-site to provide spatially resolved contamination data. The sensing device 120 may include in situ sensors for monitoring subsurface conditions, which may be used for applications involving groundwater remediation or deep soil contamination.

In some embodiments, at a treatment site, or even with a treatment device 110, there can be multiple different types of sensing devices 120, such as different types of sensors. For example, a sensing device 120 may include a direct ionization mass spectrometer, such as a Direct Analysis in Real Time Mass Spectrometer (DART-MS), which may be used to rapidly detect contaminant concentration, including PFAS and other persistent pollutants, without requiring extensive sample preparation. In some embodiments, a sensing device 120 may include a near-infrared (NIR) spectrometer, which may be used to measure soil properties such as moisture content, organic carbon levels, and mineral composition. These measurements may inform pre-processing adjustments, such as determining whether drying or chemical pre-treatment is needed before active remediation. In some embodiments, a sensing device 120 may include sensors specifically designed to measure changes in contaminant precursors, which may help determine whether additional processing is needed to fully degrade intermediate compounds.

In some embodiments, a sensing device 120 may include optical and spectral sensors, which may be used for real-time monitoring of chemical transformations occurring within the treatment device 110. The optical and spectral sensors may analyze reaction kinetics and provide feedback on whether contaminants are being effectively broken down. In some embodiments, a sensing device 120 may include thermal sensors that monitor temperature fluctuations during processing, ensuring that energy input remains within safe and efficient operating ranges. Thermal sensors may be used in applications where excess heat generation could impact chemical reaction rates, volatilize chemical compounds, or alter soil composition.

In some embodiments, a sensing device 120 may also take the form of sensors that are integrated into an in-situ monitoring system, such as sensors placed within monitoring wells for groundwater remediation. In-situ sensors may provide continuous feedback on contamination levels in subsurface environments, enabling real-time adjustments to treatment strategies.

In some embodiments, the sensing device 120 may be used in a closed-loop treatment system by providing during-treatment data that informs adjustments to the treatment process. The sensing device 120 may detect initial contaminant levels in the soil and communicate the data to a computing device 130, which may determine operational parameters. The sensing device 120 may monitor process variables while soil undergoes treatment to capture intermediate data points that allow for dynamic adaptation of treatment parameters. In some embodiments, post-treatment sensing by the sensing device 120 may verify whether contaminant levels have been reduced to target thresholds. The sensing device 120 may also facilitate regulatory compliance by providing verifiable data on contaminant reduction to provide documentation required for environmental oversight.

In some embodiments, a computing device 130 may analyze measurement data from one or more sensing devices 120, determine optimal operational parameters based on the measurements (e.g., measurements of samples from a batch of impacted soil), and control the operation of a treatment device 110, such as in a closed-loop soil remediation system. The computing device 130 may process real-time sensor data to assess soil properties, contamination levels, and treatment conditions. Based on this analysis, the computing device 130 may execute models to determine operational parameters, cost analysis, process control parameters, and whether preprocessing or post-processing is needed. A model can be rule-based algorithms, heuristic algorithms, machine learning models, such as generalized additive models (GAMs) and convolutional neural networks (CNNs), to predict treatment requirements and optimize operational parameters. The computing device 130 may communicate with a data store 140 to retrieve historical treatment data and refine predictive models over time. In some embodiments, the computing device 130 may also integrate cost-based optimization, adjusting treatment parameters to balance energy consumption and chemical additive usage in response to real-time pricing data or site-specific constraints.

Operational parameters may also be referred to as treatment parameters and may include parameters that control the treatment device 110 and parameters that adjust the treatment process. In various embodiments, operational parameters may be different, depending on the physical components of the treatment device 110 and the treatment techniques used (e.g., mechanical, chemical, or other techniques). Examples of operational parameters may include energy input, chemical dosing, dwell time, milling speed, pretreatment conditions, and post-treatment validation criteria.

In some embodiments, energy input may refer to the amount of mechanical or thermal energy applied to the soil during treatment. For example, in a ball milling treatment device 110, energy input may be controlled by adjusting the rotation speed of the milling chamber, impacting the intensity of mechanical force applied to break down contaminants. In some embodiments, energy input may also include external heating elements or electromagnetic energy sources, depending on the treatment technique used.

In some embodiments, chemical dosing may refer to the concentration and type of chemical additives introduced to accelerate contaminant degradation. For example, in some embodiments, chemical additives such as aluminum or magnesium may be introduced to enhance the oxidation of contaminants during mechanical processing. The computing device 130 may determine the optimal chemical dosing based on contaminant concentration data received from a sensing device 120 to determine the appropriate amount of reagent is used without excessive waste.

In some embodiments, dwell time may refer to the duration that soil remains in the treatment device 110 before being processed further or discharged. In some embodiment, the dwell time may be determined before the start of the active treatment based on pre-processing measurement. Alternatively, or additionally, the computing device 130 may adjust dwell time dynamically based on real-time contaminant breakdown measurements to ensure each batch of soil receives sufficient processing to meet remediation targets. In some embodiments, dwell time may range from a few minutes to several hours, depending on soil composition, contaminant type, and treatment intensity.

In some embodiments, milling speed may control the rate at which a milling reactor (e.g., ball milling) or other mechanical treatment component operates. Milling speed may influence the energy transfer within the treatment device 110, affecting how contaminants are physically broken down. Higher milling speeds may result in increased contaminant degradation rates but may also require higher energy consumption. The computing device 130 may optimize milling speed based on trade-offs between treatment efficiency and energy cost.

In some embodiments, pretreatment conditions may refer to preliminary modifications to soil before active treatment begins. Pretreatment conditions may include drying, pH adjustment, or particle size reduction, which may enhance treatment effectiveness. For example, in some embodiments, if a sensing device 120 detects high moisture content in a soil sample, the computing device 130 may initiate a drying step before introducing the soil into the treatment device 110. Similarly, in some embodiments, pH levels may be adjusted to optimize chemical reactions during treatment.

In some embodiments, post-treatment validation criteria may refer to the parameters used to determine whether treated soil meets regulatory or remediation thresholds. A sensing device 120 may measure post-treatment contaminant levels to verify that degradation has been achieved. If contaminant levels remain above target thresholds, the computing device 130 may adjust treatment parameters and initiate additional processing cycles. Post-treatment validation may also involve assessing soil stability and suitability for reintegration into the site.

In some embodiments, one or more operational parameters may be dynamically controlled by the computing device 130 in a closed-loop system to continuously refine treatment strategies based on real-time feedback from the sensing device 120. The computing device 130 may execute one or more models to enhance treatment efficiency, minimize energy and additive costs, and ensure consistent remediation performance across varying soil conditions.

In various embodiments, other examples of operational parameters may include temperature, pressure, agitation speed, gas flow rate, reactor humidity, solvent concentration, treatment cycle frequency, applied voltage, catalyst concentration, soil feed rate, discharge rate, milling intensity, energy input, chemical dosing, dwell time, pH level, oxidation-reduction potential, reaction time, moisture content, ultrasonic frequency, UV exposure time, aeration rate, microbial activity level, electromagnetic field strength, irradiation dose, adsorption capacity, extraction solvent type, filtration rate, desorption temperature, bioaugmentation agent concentration, thermal exposure duration, fluidization velocity, plasma discharge power, reagent flow rate, surfactant concentration, sonication power, mechanical shear force, etc.

In some embodiments, a computing device 130 may be implemented in various forms, depending on system requirements and deployment conditions. In some embodiments, the computing device 130 may be an onboard processing unit within a treatment device 110, enabling real-time control of mechanical and chemical treatment parameters. In some embodiments, the computing device 130 may be a device located onsite. An onsite computing device 130 may collect and analyze data from multiple treatment devices 110 deployed across a contaminated site. In some embodiments, an onsite computing device 130 may simply be a computer, a tablet, or a smartphone that is installed with a software application that includes the models used to communicate with a sensing device 120 and control a treatment device 110. For example, producers of the treatment device 110 may publish a software application for an operator onsite to down the software into the operator's phone or tablet. In some embodiments, the computing device 130 may be a remote computing server, such as a server that leverages cloud-based computing resources, storing large datasets in a data store 140 and executing computationally intensive machine learning algorithms remotely. The computing device 130 may be equipped with interfaces for remote monitoring and control, allowing operators to adjust treatment parameters, review contamination levels, and ensure compliance with regulatory requirements. In some embodiments, the computing device 130 may also support integration with external environmental monitoring systems to enable real-time coordination with broader site remediation efforts.

In some embodiments, a computing device 130 may function as the controller in a closed-loop soil treatment system, which may continuously refine treatment strategies based on sensor feedback during the treatment process. The computing device 130 may receive initial contaminant concentration data from a sensing device 120 before treatment begins, determining the appropriate energy input, additive dosing, and processing time required for effective remediation. As the soil undergoes treatment within a treatment device 110, the computing device 130 may analyze intermediate sensor readings to adjust parameters dynamically to monitor whether contaminants are degraded accordingly to predicted modeling of the degradation. If post-treatment analysis by the sensing device 120 indicates that contamination levels remain above target thresholds, the computing device 130 may automatically trigger additional treatment cycles or modify processing conditions.

The computing device 130 may continuously refine treatment parameters based on historical treatment data stored in a data store 140, improving remediation efficiency over time. The treatment device 110 may support adaptive energy and chemical dosing strategies based on site-specific constraints, such as cost considerations and regulatory compliance requirements. In some embodiments, the computing device 130 may utilize deep learning techniques, such as reinforcement learning, to improve remediation outcomes over time and update predictive models based on past treatment data stored in the data store 140. In some embodiments, the treatment device 110 may enable precise remediation of heterogeneous contamination levels within a site by dynamically tailoring treatment conditions to each soil batch, reducing resource consumption while ensuring effective contaminant degradation.

In some embodiments, a computing device 130 may be used regardless of whether the treatment occurs in-situ or ex-situ. In an ex-situ configuration, the treatment device 110 may receive excavated soil batches, process them under optimized treatment conditions, and verify contaminant degradation through post-treatment analysis. In an in-situ configuration, the treatment device 110 may be integrated with monitoring wells or subsurface injection systems to introduce treatment agents and track contaminant reduction in real time. The computing device 130 may receive feedback from a sensing device 120 to adjust treatment conditions dynamically, ensuring that remediation targets are met while optimizing resource usage. In some embodiments, historical treatment data stored in a data store 140 may be used to refine in-situ or ex-situ treatment strategies over time, enhancing efficiency across multiple remediation projects.

In some embodiments, a data store 140 may be a storage system that is used to collect, manage, and retrieve data related to the soil treatment process. The data store 140 may store sensor data from a sensing device 120, operational parameters determined by a computing device 130, and historical records of soil remediation outcomes. The data store 140 may retain information such as contaminant concentrations before and after treatment, operational parameters used for each batch of soil, and adjustments made by the computing device 130 during processing. In some embodiments, the data store 140 may facilitate regulatory compliance by maintaining a record of treatment performance, ensuring that remediation meets required environmental standards. The data store 140 may provide data to support analytics and reporting functions, allowing for trend analysis, process optimization, and predictive modeling based on past treatment data.

In some embodiments, the data store 140 may be implemented in various forms, depending on system architecture and operational requirements. In some embodiments, the data store 140 may be a local storage system, such as an onboard memory within the computing device 130 or within a treatment device 110, allowing immediate access to treatment data during active remediation. In some embodiments, the data store 140 may be a centralized database located on-site, collecting and processing data from multiple treatment devices 110 operating simultaneously. In some embodiments, the data store 140 may be a cloud-based storage system that allows remote access to historical and real-time data for monitoring, analysis, and machine learning model training. The data store 140 may support encrypted data transmission and secure access protocols to ensure the integrity and confidentiality of remediation records. In some embodiments, the data store 140 may integrate with external regulatory databases, providing automated reporting capabilities for environmental agencies and stakeholders. The data store 140 may also be connected to a local power supplier to receive energy cost data from the power supplier. The energy cost data may vary based on dates and time and the computing device 130 may adjust operational parameters based on energy cost.

In some embodiments, the data store 140 may provide data in a closed-loop soil treatment system, such as in continuous learning and optimization of remediation strategies. The computing device 130 may access historical data stored in the data store 140 to refine machine learning models and improve treatment efficiency. For example, the computing device 130 may analyze past treatment records to identify correlations between soil composition and optimal processing parameters, allowing for more precise adjustments in future remediation efforts. In some embodiments, the data store 140 may store cost-related data, including energy consumption, chemical additive usage, and equipment wear, enabling cost-benefit analysis and process optimization. The data store 140 may also facilitate anomaly detection by tracking deviations in treatment performance, allowing for early identification of equipment malfunctions or unexpected contaminant behavior. In some embodiments, the data store 140 may provide real-time access to operators, enabling remote diagnostics and process control adjustments. Through integration the data store 140, the computing device 130, in some embodiments, may support an automated, adaptive, and data-driven approach to soil remediation, enhancing efficiency, reliability, and compliance with environmental standards. While some embodiments are described as automated, the computing device 130 may additionally allow human-in-the-loop feedback and controls for the treatment device 110.

The communications among the components in the system environment 100 may be through direct communication on site or may be transmitted via a network. In some situations, a network may be a local network. In some situations, a network may be a public network such as the Internet. In one embodiment, the network uses standard communications technologies and/or protocols. Thus, the network can include links using technologies such as Ethernet, 802.11, worldwide interoperability for microwave access (WiMAX), 3G, 4G, LTE, 5G, digital subscriber line (DSL), asynchronous transfer mode (ATM), InfiniBand, PCI Express Advanced Switching, etc. Similarly, the networking protocols used on the network can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), etc. The data exchanged over the network can be represented using technologies and/or formats, including the hypertext markup language (HTML), the extensible markup language (XML), etc. In addition, all or some of the links can be encrypted using conventional encryption technologies such as secure sockets layer (SSL), transport layer security (TLS), virtual private networks (VPNs), Internet Protocol security (IPsec), etc. The network also includes links and packet-switching networks such as the Internet.

### Example Treatment Device

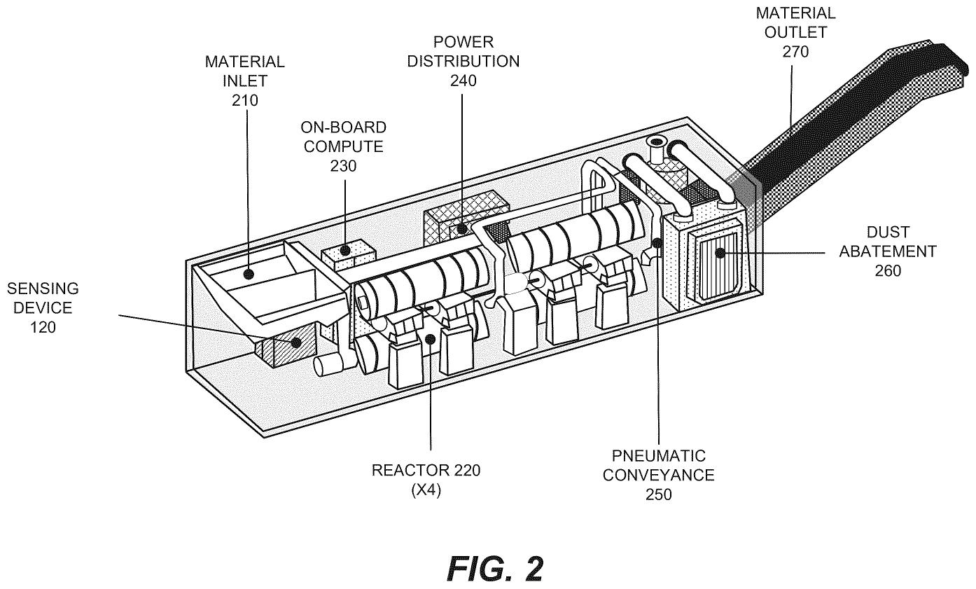

FIG. 2 is a perspective view of a treatment device 200, in accordance with some embodiments. The treatment device 200 may be an example of a treatment device 110 described in FIG. 1 that is used for treating PFAS contaminated soil. In various embodiments, the components of a treatment device 110 may change, based on the treatment techniques, contaminants, site requirements and other techniques. The components and physical arrangement of the treatment device 200 illustrated in FIG. 2 are merely an example. The 200 may include a material inlet 210, a sensing device 120, a reactor 220, an on-board compute 230, a power distributor 240, a pneumatic conveyance 250, a dust abatement 260, and a material outlet 270. in some embodiments, the treatment device 200 may include additional, fewer, or different components.

In some embodiments, the material inlet 210 may be configured to receive impacted soil for processing within the treatment device 200. The material inlet 210 may be positioned at the initial stage of the treatment process to facilitate a controlled introduction of impacted soil into the treatment device 200. The material inlet 210 may include a conveyance mechanism, such as a hopper, chute, or automated feeder, that regulates the flow of material into the treatment device 200. In some embodiments, the material inlet 210 may be configured to interface with a mechanical conveyance component for efficient transfer of soil into the reactor 220. In some embodiments, the material inlet 210 may include a sealing or containment mechanism to prevent dust dispersion or contamination escape during loading. The material inlet 210 may also incorporate automated flow control systems, such as variable-speed feeders or gate valves, to regulate the rate of soil entry based on real-time feedback from the computing device 130.

In some embodiments, the material inlet 210 may support batch or continuous processing modes. For batch processing, the material inlet 210 may be designed to receive discrete soil batches to allow for individualized treatment optimization per batch. For continuous processing, the material inlet 210 may facilitate a steady flow of material, synchronized with real-time adjustments in treatment parameters determined by the computing device 130. The material inlet 210 may be adaptable to various soil consistencies, including dry, damp, or sludge-like materials. The dimensions and shape of the material inlet 210 may be adapted based on site-specific requirements, such as the volume and composition of soil being treated.

In some embodiments, the material inlet 210 may be integrated with the sensing device 120 to enable pre-treatment and/or real-time analysis of incoming soil. The sensing device 120 may be positioned proximate to the material inlet 210 to capture contamination levels, moisture content, or other relevant soil properties prior to treatment. The sensing device 120 may utilize one or more analytical techniques.

In some embodiments, the sensing device 120 may utilize one or more analytical techniques and may include a combination of analytical sensors configured to measure various soil properties. Analytical techniques may include direct ionization mass spectrometry, near-infrared spectroscopy, optical spectroscopy, thermal imaging, or other spectral analysis methods to determine soil characteristics and inform subsequent treatment parameters. The sensing device 120 may be configured to detect contamination levels, including the presence and concentration of PFAS or other hazardous compounds, as well as soil composition factors such as organic carbon content, mineral composition, and particle size distribution. For example, the sensing device 120 may incorporate a direct ionization mass spectrometer, such as a Direct Analysis in Real-Time mass spectrometry (DART-MS) system, to detect contamination levels, including PFAS and other hazardous compounds. The sensing device 120 may further include near-infrared (NIR) spectroscopy sensors for analyzing organic carbon content, mineral composition, and moisture levels. Optical and spectral sensors may be included to monitor chemical transformations and reaction progress.

The sensing device 120 may further incorporate moisture sensors, pH sensors, or conductivity probes to assess pre-treatment conditions that may influence the efficacy of the treatment process. Thermal sensors may be integrated to measure soil temperature variations that could impact treatment efficiency. The sensing device 120 may also feature pH and conductivity sensors to assess soil acidity and ionic content, which may influence chemical additive performance. Additionally, imaging sensors, such as hyperspectral cameras or laser-induced breakdown spectroscopy (LIBS), may be used to provide detailed compositional mapping of soil samples.

In some embodiments, the sensing device 120 may be modular and include multiple sensors, depending on the specific treatment application. Some sensors may be positioned directly at the material inlet 210 to analyze incoming soil in real time, while others may be housed in the reactor 220 or at a conveyance component, where soil samples are periodically analyzed. A sensing device 120 may also be designed to withstand environmental factors such as dust, vibration, and temperature fluctuations.

In some embodiments, the sensing device 120 in FIG. 2 may include a sample collection device for collecting samples to be analyzed at a sensing device 120 (e.g., a laboratory station) positioned outside the treatment device 200 for on-site measurement. The sensing device 120 may incorporate mechanical arms or robotic actuators configured to excavate soil samples from various locations at a remediation site. The sensing device 120 may then transfer the collected samples to an onsite analysis station, where contamination levels, moisture content, and other soil properties are measured. The sensing device 120 may operate autonomously or under remote control to allow sample collection during a treatment process to refine operational parameters.

In some embodiments, the reactor 220 may be configured to facilitate the physical and/or chemical treatment of contaminated soil through a combination of mechanical agitation, chemical co-addition, and controlled processing conditions. In some embodiments, the reactor 220 may carry out a mechanochemical operation. For example, the reactor 220 may include a high-energy ball milling chamber, wherein contaminated soil is subjected to intense grinding forces generated by rotating agitator blades and impact media, such as steel or ceramic balls. The reactor 220 may operate at varying energy densities, rotational speeds, and dwell times. These operational parameters may be dynamically adjustable based on sample measurements provided by one or more sensing devices 120.

In some embodiments, the reactor 220 may be configured to operate at temperatures below 50 degrees Celsius (e.g., room temperature) and at pressures below 1.5 times atmospheric pressure (e.g., at atmospheric pressure with gas outlet that allows the control of the pressure). Operating within these parameters may facilitate easy sample collection during and after treatment, as soil conditions remain stable and do not require specialized high-temperature or high-pressure containment measures. The controlled temperature and pressure conditions may further enable the operation of certain types of sensors within or proximate to the reactor 220, including thermal sensors, optical spectroscopy sensors, and direct ionization mass spectrometry devices, which may provide real-time feedback on treatment progress. In some embodiments, maintaining a low-temperature and near-atmospheric pressure environment may prevent the volatilization of hazardous compounds, reducing the risk of toxic gas release and ensuring that contaminants, such as PFAS, undergo controlled degradation rather than being emitted into the surrounding environment.

The operation temperature for the reactor 220 may vary in different embodiments. In some embodiments, the reactor 220 may be configured to operate at temperatures below 50 degrees Celsius. In some embodiments, the reactor 220 may be configured to operate at temperatures below 60 degrees Celsius. In some embodiments, the reactor 220 may be configured to operate at temperatures below 70 degrees Celsius. In some embodiments, the reactor 220 may be configured to operate at temperatures below 80 degrees Celsius. In some embodiments, the reactor 220 may be configured to operate at temperatures below 90 degrees Celsius. In some embodiments, the reactor 220 may be configured to operate at temperatures below 100 degrees Celsius. In some embodiments, the reactor 220 may be configured to operate at temperatures below 110 degrees Celsius. In some embodiments, the reactor 220 may be configured to operate at temperatures below 120 degrees Celsius. In some embodiments, the reactor 220 may be configured to operate at temperatures below 130 degrees Celsius. In some embodiments, the reactor 220 may be configured to operate at temperatures below 140 degrees Celsius. In some embodiments, the reactor 220 may be configured to operate at temperatures below 150 degrees Celsius.

In some embodiments, the reactor 220 may be a custom ball mill configured for mechanochemical treatment of PFAS-impacted soil. The reactor 220 may utilize a stirred rotor architecture, wherein a central agitator is driven by a permanent magnet AC (PMAC) motor to facilitate high-energy milling. In some embodiments, energy input to the reactor 220 may be adjustable across a range of power-to-volume ratios, allowing for optimized throughput and energy efficiency. Experimental evaluations conducted at varying energy scales indicate that reactors with higher power-to-volume ratios may achieve both increased processing capacity and improved energy efficiency. In some embodiments, commercially available ball mills designed for particle size reduction may lack the required energy densities for effective PFAS degradation, necessitating a custom reactor design. The reactor 220 may include robust sealing mechanisms and thermal management features to maintain stable operating conditions. Additionally, the reactor 220 may be configured to integrate various sensors from the sensing device 120 to enable real-time monitoring of treatment conditions.

In some embodiments, the reactor 220 may introduce a non-toxic additive to facilitate the degradation of PFAS compounds during treatment. The computing device 130 may dynamically adjust the amount and timing of additive introduction based on optimization algorithms that account for real-time soil properties and process efficiency. Unlike prior mechanochemical treatment systems, which rely on fixed dosing strategies, the reactor 220 may modify additive concentrations adaptively in response to live sensor feedback. In some embodiments, the reactor 220 may utilize additives that do not require post-milling neutralization or secondary treatment. The selection of an optimal additive formulation may depend on multiple factors, including site-specific soil composition, electricity cost, and market pricing of chemical reagents. The computing device 130 may analyze these variables to determine a cost-effective and efficient additive application strategy for a given soil batch.

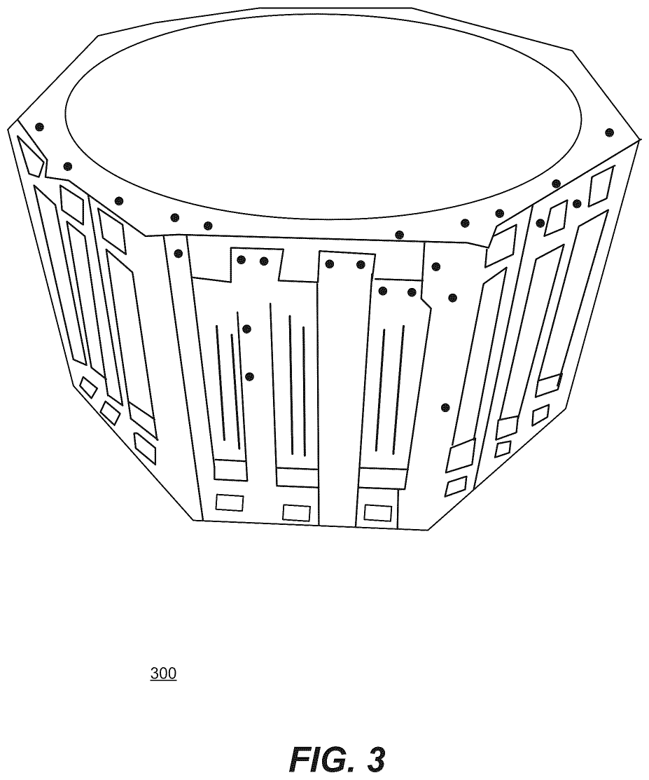

Referring temporarily to FIG. 3, a perspective view of an example ball milling chamber 300 is illustrated, in accordance with some embodiments. The ball milling chamber 300 is an example of a reactor 220. In some embodiments, the ball milling chamber 300 may facilitate high-energy mechanical processing of impacted soil. The internal structure of the ball milling chamber 300 may include a cylindrical or conical chamber lined with wear-resistant materials such as hardened steel, ceramic, or tungsten carbide to withstand prolonged high-energy impacts. The ball milling chamber 300 may house a central rotor or multiple agitator arms configured to impart kinetic energy to grinding media, such as steel or ceramic balls. The grinding media may be of varying sizes and compositions to optimize impact force and shearing action, enhancing the breakdown of contaminants. The ball milling chamber 300 may be enclosed with a sealed access hatch to prevent material loss and control airborne particulates. In some embodiments, the ball milling chamber 300 may include internal baffles or partitions to ensure even distribution of soil throughout the milling process.

In some embodiments, the ball milling chamber 300 may incorporate additional subsystems to optimize treatment conditions. For example, the ball milling chamber 300 may include an adjustable-speed motor to control rotational velocity, allowing for variation in milling intensity based on soil properties and contamination levels. The ball milling chamber 300 may further include internal thermal sensors to monitor temperature fluctuations to maintain the operational limits to below a certain threshold (e.g., 50 degrees Celsius) to prevent volatilization of hazardous compounds. The ball milling chamber 300 may also include chemical additive injection ports to allow for addition and adjustment of chemical agents, such as aluminum or magnesium, to enhance contaminant degradation. The ball milling chamber 300 may also include an automated discharge mechanism that regulates the removal of processed material through the material outlet 270 while preventing cross-contamination between batches.

In some embodiments, the ball milling chamber may be one example of a reactor type, and other configurations may also be implemented depending on site conditions, soil composition, and contaminant characteristics. Alternative or additional reactor types may include attritor mills that utilize high-speed stirred media for fine grinding and enhanced reaction kinetics. Alternatively, or additionally, reactors 220 may include fluidized bed reactors that suspend soil particles in a chemically reactive gas or liquid flow to promote contaminant breakdown. Alternatively, or additionally, reactors 220 may include ultrasonic cavitation reactors that apply high-frequency acoustic energy to generate localized high-pressure zones that enhance chemical degradation. Alternatively, or additionally, reactors 220 may include plasma reactors that use ionized gas to break down complex contaminants through high-energy oxidation. The reactor 220 may also incorporate hybrid designs that combine mechanical processing with chemical or thermal enhancements to enhance treatment efficiency.

Referring back to FIG. 2, in some embodiments, the reactor 220 may incorporate multiple milling chambers arranged in series or parallel configurations to increase throughput and optimize energy efficiency. The reactor 220 may include modular treatment zones, each of which may be independently controlled to apply specific chemical or mechanical conditions tailored to the soil properties of a given batch. For example, each chamber of a reactor 220 may include individual internal sensing devices 120 to monitor parameters such as temperature, pressure, reaction kinetics, contaminant level to provide measurement data to a computing device 130 to determine chamber-specific operational parameters.

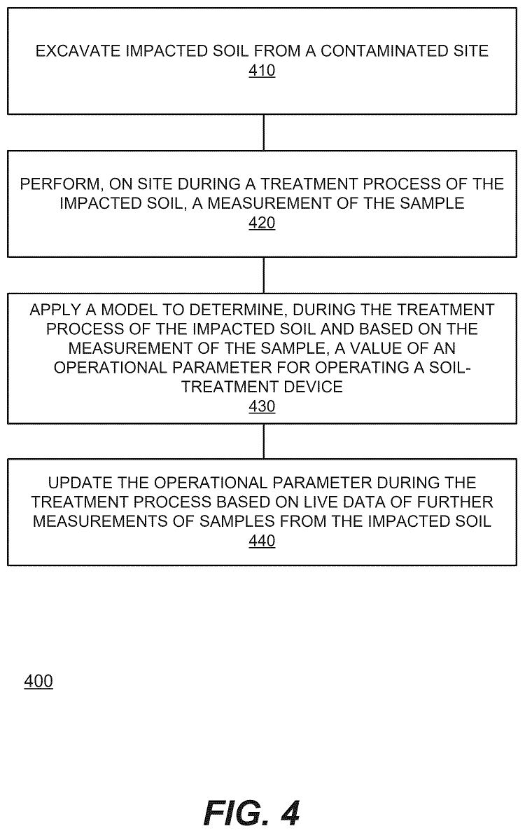

In some embodiments, various sensing devices 120, whether they are at the material inlet 210 or inside a treatment device 200, may be integrated with an on-board compute 230 to enable real-time data processing and dynamic adjustment of treatment parameters. The on-board compute 230 may be an example of computing device 130. In some embodiments, the treatment device 200 may not include the on-board compute 230 but instead transmit the measurement data to a computing device 130 outside of the treatment device 200. A sensing device 120 may transmit (e.g., continuously) measurement data to the on-board compute 230, which may analyze contamination trends, evaluate soil variability, and determine operational parameters such as dwell time, chemical additive concentration, and milling intensity in one or more reactors 220. A sensing device 120 and the on-board compute 230 may provide a feedback control mechanism for the current batches and future batches. Examples of detail of modeling and algorithms that may be used in an on-board compute 230 (or any computing device 130) are further discussed in FIG. 4 through FIG. 8.

In some embodiments, the power distributor 240 may be configured to manage and regulate the distribution of electrical power to various components of the treatment device 200. The power distributor 240 may receive power from an external power source, such as a grid connection, generator, or battery system, and allocate power to one or more reactors 220, the sensing device 120, the on-board compute 230, pneumatic conveyance 250, and dust abatement 260 based on real-time operational demands. The power distributor 240 may include voltage regulators, circuit breakers, and power monitoring systems to ensure stable and efficient energy supply to all connected subsystems.

In some embodiments, the power distributor 240 may dynamically adjust power allocation among the reactors 220 based on feedback from a computing device 130 (e.g., the on-board compute 230), which may optimize power consumption according to processing requirements. For example, the power distributor 240 may prioritize energy delivery to the reactor 220 during high-energy treatment phases while reducing power to non-essential subsystems. The power distributor 240 may also support integration with renewable energy sources, such as solar or wind power, for enhanced energy efficiency and environmental sustainability.

In some embodiments, the pneumatic conveyance 250 may be configured to transport soil between different stages of the treatment process within the treatment device 200. The pneumatic conveyance 250 may utilize compressed air or vacuum-driven systems to move soil from the material inlet 210 to the reactor 220 and from the reactor 220 to the material outlet 270. The pneumatic conveyance 250 may be designed to handle varying soil consistencies, including dry, damp, or fine particulate material. The pneumatic conveyance 250 may also integrate flow control mechanisms to regulate transfer speed and prevent clogging or material buildup.

In some embodiments, the dust abatement 260 may be configured to reduce airborne particulate emissions generated during soil processing within the treatment device 200. The dust abatement 260 may include filtration systems, such as high-efficiency particulate air (HEPA) filters, cyclone separators, or water misting systems, to capture and contain dust. The dust abatement 260 may operate in coordination with pneumatic conveyance 250 to maintain air quality and prevent soil loss during material transfer. The dust abatement 260 may further include automated monitoring sensors to detect dust levels and dynamically adjust mitigation measures for optimal environmental control.

In some embodiments, the material outlet 270 may be configured to discharge treated soil from the reactor 220 after processing is complete. The material outlet 270 may include an automated gate, conveyor system, or controlled chute to regulate the release of soil while preventing cross-contamination between batches. The material outlet 270 may be designed to accommodate real-time sampling, allowing post-treatment verification by the sensing device 120 before soil is removed. The material outlet 270 may further integrate with external handling systems, such as storage containers or transport conveyors, to facilitate efficient removal and site management of treated material.

In some embodiments, the material outlet 270 may be configured to discharge treated soil from the reactor 220 while incorporating additional sensors for post-processing analysis and treatment verification. The material outlet 270 may include sensors such as direct ionization mass spectrometry devices, near-infrared spectroscopy sensors, or optical and thermal sensors to measure residual contamination levels, moisture content, and chemical composition before the soil exits the treatment device 200. The material outlet 270 may further integrate automated sampling mechanisms that collect treated soil for external validation. The material outlet 270 may interface with external transport systems to export verified, fully treated soil and retain soil that needs further treatment. For example, the material outlet 270 may include a reverse component to put soil back to one of the reactors 220.

### Example Treatment Process

FIG. 4 is a flowchart depicting a soil treatment process 400, in accordance with some embodiments. FIG. 4 may be performed automatically by a computing device 130 controlling a treatment device 110 or be performed partially with human intervention. For example, sample collection may be performed automatically through on-board sensors, automatically by robotic arms, or manually through an operator collecting samples periodically during the treatment process. Likewise, the operational parameters may be set by a computing device 130 in a fully automated manner or be set through a human-in-the-loop process, where an operator may provide decisions based on various outputs and predictions generated by the computing device 130. As such, while some of the steps are described as being performed by the computing device 130, if applicable, the steps may also be performed at least partially by an operator. In various embodiments, the soil treatment process 400 may include additional, fewer, or different steps. The steps in the soil treatment process 400 may also be performed in a different order than the illustration shown in FIG. 4.

While the process 400 is described with the treatment of PFAS-impacted soil, the process 400 may also be used in the treatment of other types of contaminants and with other treatment techniques. Variations of different treatments and techniques are discussed in other parts of this disclosure, such as in discussion associated with FIG. 1.

In some embodiments, the computing device 130 may cause 410 an excavation of impacted soil from a contaminated site. The impacted soil may be PFAS-impacted soil and the contaminated site may be PFAS-contaminated site. The excavation process may be performed using as part of an ex-situ treatment process. The excavated soil may then be transferred to a designated a material inlet 210 of a treatment device 110 before treatment.

In some embodiments, the computing device 130 may perform 420, on site during a treatment process of the PFAS-impacted soil, a measurement of the sample. A sensing device 120 may be configured to analyze various parameters of the soil sample, including contaminant concentration, moisture content, organic carbon levels, mineral composition, and temperature. Multiple measurements may be taken. For example, the measurement process may occur as part of the pre-treatment and also during the active treatment, which allows for continuous monitoring of soil properties throughout the treatment process.

For example, in pre-treatment, the excavated PFAS-impacted soil may be analyzed by an initial characterization process to determine relevant soil properties, including contaminant concentration, moisture content, organic carbon levels, and mineral composition. The characterization process may involve real-time sensor analysis or laboratory-based measurement techniques. The sensing device 120 may include various analytical instruments, such as a direct ionization mass spectrometer, near-infrared (NIR) spectroscopy sensor, thermal sensor, or optical and spectral sensors, to provide accurate contaminant profiling. In some embodiments, the excavation process may be designed to accommodate variability in contamination levels across different soil batches. PFAS contamination at a single site may vary by orders of magnitude between different locations. The computing device 130 may determine batch-specific treatment strategies by analyzing initial soil properties prior to initiating the treatment process. The initial analysis process may also account for factors such as soil texture, clay content, and entrained water, which may influence the effectiveness of subsequent remediation steps.

In some embodiments, pre-treatment steps may be performed on the excavated soil to optimize treatment efficiency. These pre-treatment steps may include drying, pH adjustment, or the addition of chemical additives to enhance the mechanochemical reaction process. The computing device 130 may determine whether pre-treatment steps are needed for a particular batch based on initial soil characterization data and real-time analysis from the sensing device 120.

In some embodiments, the computing device 130 may analyze PFAS precursors present in the PFAS-impacted soil to optimize the treatment process. PFAS precursors are compounds that may degrade into detectable PFAS species during treatment, influencing the overall degradation rate and treatment efficiency. The sensing device 120 may be configured to conduct measurements such as total organic fluorine. The computing device 130 may compute estimates for parameters that indicate the presence of PFAS precursors such as by providing estimates based on total organic fluorine and other specific PFAS analytes. The computing device may determine the estimates via models and algorithms that are generated using data from other contaminated sites. The ratio of precursors to terminal PFAS compounds may vary across different soil batches, requiring an adaptive treatment approach. The presence of precursors may slow down degradation kinetics. The computing device 130 may adjust operational parameters to account for the precursors.

Subsequent to pre-treatment, an active treatment process may begin. In some embodiments, to perform an active treatment, the computing device 130 may use a treatment device 110. The treatment device 110 may include a reactor, such as a ball mill reactor, that facilitates the mechanochemical degradation of PFAS-impacted soil. The ball mill reactor may comprise a rotary shaft and metallic ball bearings, which interact with the soil through high-energy milling. The rotational speed of the reactor may be one of the operational parameters that can be adjusted based on the measurements performed by the sensing device 120.

In some embodiments, the reactor may be configured to operate at a controlled temperature and pressure. The computing device 130 may regulate the reactor to maintain a temperature below 50 degrees Celsius and below 1.5 times atmospheric pressure, which allow the treatment process to remain energy efficient while avoiding volatilization of contaminants. The sensing device 120 may include thermal sensors to continuously monitor temperature conditions within the reactor and provide feedback to the computing device 130.

In some embodiments, the reactor may receive chemical additives, such as aluminum and magnesium metallic additives, to enhance the mechanochemical degradation process. The amount of metallic additive introduced into the reactor may be determined based on soil composition and contamination levels. The computing device 130 may adjust the additive dosage dynamically, optimizing degradation efficiency while minimizing excess material use.

In some embodiments, the sensing device 120 may take different forms. For example, a sensing device 120 may be an automatic sensor installed within the treatment device 110. This type of sensors may allow real-time process monitoring. The automatic sensor may be embedded within the reactor chamber to directly measure soil parameters during the milling operation. The sensing device 120 may include a direct ionization mass spectrometer, a near-infrared (NIR) spectroscopy sensor, a thermal sensor, or optical and spectral sensors to capture real-time analytical data. In some embodiments, the sensing device 120 may also be a separate measurement device positioned outside the treatment device 110. The sensing device 120 may include laboratory-based measurement systems, such as soil analysis kits, which provide additional verification of contaminant degradation. The computing device 130 may integrate data from both in-line sensors and external laboratory measurements to refine process optimization.

In some embodiments, the computing device 130 may apply 430 a model to determine, during the treatment process of the PFAS-impacted soil and based on the measurement of the sample, a value of an operational parameter for operating a soil-treatment device. The operational parameter may be dynamically adjusted based on real-time measurements from the sensing device 120 to optimize the mechanochemical treatment process. The model applied by the computing device 130 may be a rule-based model, a heuristic-based model, or a machine learning model, depending on the complexity of the treatment optimization required.

In some embodiments, the model may be a rule-based model that applies predefined decision rules to determine operational parameters. For example, the computing device 130 may implement a lookup table that correlates measured PFAS concentrations with specific milling speeds and additive dosing requirements. If the sensing device 120 detects a contamination level exceeding a predefined threshold, the rule-based model may automatically increase the milling speed or extend the dwell time to enhance degradation efficiency. Similarly, if the moisture content of the soil is above a certain level, the model may trigger a pre-treatment drying step before initiating the mechanochemical process.

In some embodiments, the model may be a heuristic-based model that applies empirical relationships or proxies to estimate optimal operational parameters. For example, instead of directly measuring PFAS degradation rates, the computing device 130 may use temperature rise within the reactor as a proxy for energy input and reaction progress. If the temperature remains below a threshold despite continued milling, the model may infer that additional energy input or chemical additives are required. Similarly, the model may use the presence of specific mineral compositions in the soil as an indicator of potential PFAS precursor transformation rates, adjusting treatment conditions accordingly. The heuristic-based model may also incorporate cost considerations, such as selecting energy-intensive processing only when cheaper grid power is available and relying on chemical additives when power costs are high.

In some embodiments, the model may be a machine learning model configured to analyze complex contamination patterns and optimize operational parameters dynamically. The computing device 130 may use a Generalized Additive Model (GAM) to predict necessary treatment adjustments based on multiple input factors, such as soil composition, PFAS precursor concentrations, and milling conditions. The GAM may regress the influence of each process parameter independently, allowing for flexible, non-linear optimization of treatment conditions. In some embodiments, the computing device 130 may use a convolutional neural network (CNN) to analyze hyperspectral imaging data from NIR spectroscopy. The CNN may detect hidden features in soil composition and contamination patterns that are not easily discernible through traditional sensing techniques. For example, the CNN may identify spectral signatures associated with PFAS precursor transformation and adjust milling speed or additive dosing accordingly. The machine learning model may also refine its predictions over time by incorporating feedback from post-treatment contaminant measurements, improving the accuracy of future treatment optimizations.

In some embodiments, the computing device 130 may integrate multiple models within a hybrid framework to enhance treatment efficiency. A rule-based model may handle baseline parameter settings for routine operations, while a heuristic-based model may refine adjustments based on real-time reactor conditions. Machine learning models may provide continuous optimization by identifying trends and patterns in historical data, allowing the system to improve over time. By combining these approaches, the computing device 130 may ensure that treatment parameters are adapted dynamically to achieve optimal PFAS degradation while minimizing energy and additive consumption.

In some embodiments, real-time sensing and data analysis may enable batch-specific treatment strategies. PFAS-impacted soil from a contaminated site may have highly variable contamination levels between batches. The computing device 130 may use machine learning models to process measurement data and adapt treatment conditions accordingly. This closed-loop optimization approach may reduce treatment time, lower energy consumption, and improve overall remediation effectiveness.

In some embodiments, the computing device 130 may apply the determined operational parameter to the treatment device 110, which carries out the treatment process that includes a mechanochemical operation to treat the PFAS-impacted soil according to the operational parameter. The mechanochemical operation may involve high-energy milling with metallic ball bearings, chemical additive reactions, and controlled temperature and pressure conditions. The treatment process may be continuously monitored by the sensing device 120, and the computing device 130 may update operational parameters in real time based on live data to maintain optimal performance.

In some embodiments, the computing device 130 may update 440 the operational parameter during the treatment process based on live data of further measurements of samples from the PFAS-impacted soil. The sensing device 120 may perform continuous or periodic measurements to assess the ongoing progress of the treatment process, capturing data such as PFAS degradation levels, temperature, milling energy, chemical additive concentration, and reaction byproducts. The computing device 130 may process these measurements in real time to refine the treatment strategy dynamically.

In some embodiments, the computing device 130 may determine an estimated operational parameter based on soil parameters measured before and during treatment. The computing device 130 may use an optimization algorithm to compare estimated degradation progress with target treatment end points. If the sensing device 120 detects lower-than-expected PFAS degradation, the computing device 130 may adjust operational parameters such as increasing the milling speed, extending dwell time, or modifying the chemical additive dosage to enhance the treatment efficiency.

In some embodiments, the computing device 130 may calculate an estimated destruction rate of PFAS compounds in real time. The estimated destruction rate may be determined using mathematical models that incorporate measured soil properties, historical treatment data, and ongoing reactor conditions. The computing device 130 may use this estimated destruction rate to ensure that the treatment process remains within an optimized efficiency range, preventing under-treatment or excessive energy and additive consumption.

In some embodiments, the computing device 130 may identify a target treatment end point based on regulatory or performance thresholds. The target treatment end point may correspond to a predefined contaminant concentration level, ensuring that the treated soil meets environmental compliance requirements. The computing device 130 may continuously compare live measurement data against the target treatment end point and adjust the treatment strategy dynamically to maintain process efficiency.

In some embodiments, the computing device 130 may provide the estimated destruction rate, the target treatment end point, and the estimated operational parameter into an optimization algorithm to adjust the operational parameter based on cost considerations. The optimization algorithm may evaluate cost trade-offs between energy input and chemical additive usage, ensuring that treatment efficiency is maintained while minimizing resource expenditure. For example, if energy costs are high at a given time, the computing device 130 may prioritize chemical additive adjustments over increased milling energy. Conversely, if chemical additive costs exceed a threshold, the computing device 130 may optimize treatment using higher mechanical energy input instead.

In some embodiments, the computing device 130 may output a recommended action based on real-time analysis of treatment performance. The recommended action may include a post-treatment step or an updated value of an operational parameter. A post-treatment step may include secondary milling or additional chemical amendments to achieve complete degradation of PFAS compounds. The computing device 130 may also adjust the operational parameters for subsequent soil batches based on insights gained from prior treatments, ensuring continuous process improvement.

In some embodiments, the computing device 130 may incorporate live energy cost data into the optimization process. The computing device 130 may adjust operational parameters in response to fluctuations in energy pricing, selecting cost-efficient treatment strategies in real time. For example, if real-time power costs decrease, the computing device 130 may increase milling energy to accelerate treatment. If energy costs rise, the computing device 130 may compensate by optimizing chemical additive use instead.

In some embodiments, the computing device 130 may leverage historical treatment data and machine learning models to refine real-time adjustments. By continuously learning from past treatment cycles, the computing device 130 may improve its predictions for optimal operational parameters, reducing processing time and enhancing cost efficiency. This iterative learning approach may enhance the overall effectiveness of the closed-loop soil treatment system.

### Data Analysis Pipeline

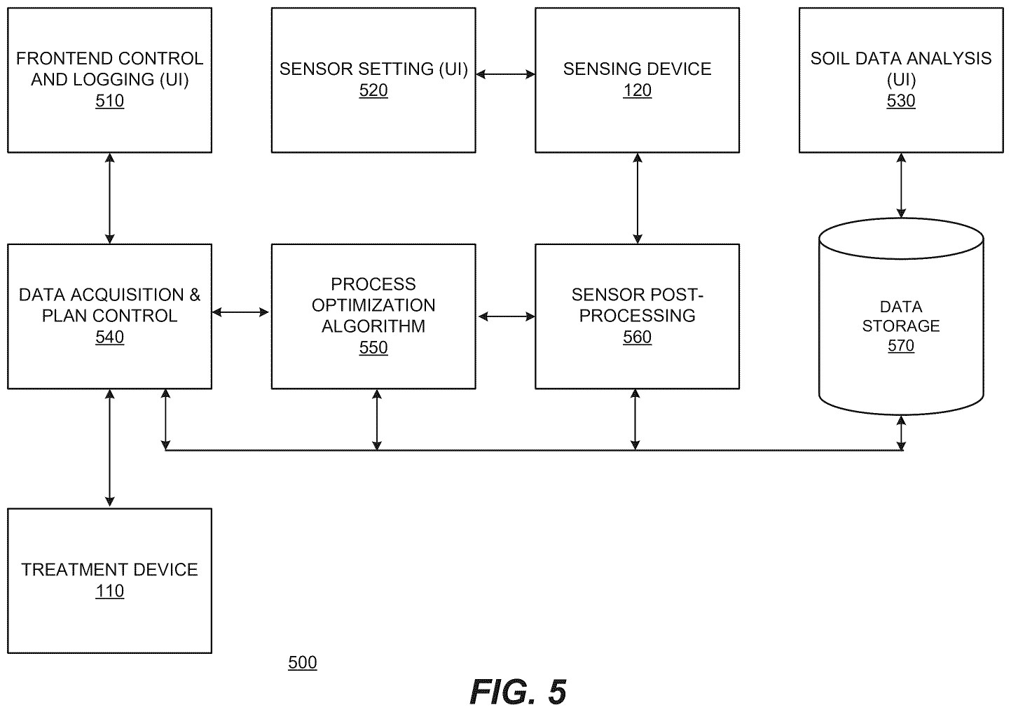

FIG. 5 is a block diagram depicting a data processing pipeline 500, in accordance with some embodiments. The data processing pipeline 500 may be performed by any suitable computing device, such as the computing device 130. In various embodiments, a data processing pipeline 500 may include fewer, additional, or different steps that are described in FIG. 5.

In some embodiments, the computing device 130 may perform seven sequential stages to facilitate a closed-loop soil treatment process, integrating in-process sensing, process control, optimization algorithms, and cloud-based data management. These stages may include system control, sensor interfacing, data acquisition, process optimization, and cloud storage to improve efficient PFAS remediation.

In some embodiments, the computing device 130 may execute a frontend control and process logging stage 510 to allow user interaction with a treatment device 110. The frontend interface may take the form of a graphical user interface (GUI). The interface may enable users to select operational parameters, configure sample processing conditions, and visualize system performance. The computing device 130 may provide a graphical representation of soil contamination levels, system alerts, and treatment progress. The user interface may provide real-time monitoring and human-in-the-loop manual overrides as needed.

In some embodiments, in stage 520, the computing device 130 may provide a user interface for adjusting sensor hardware to configure and control one or more sensing devices 120 to capture real-time soil properties and contamination levels. The sensing devices 120 may include mass spectrometers, near-infrared (NIR) spectroscopy, thermal sensors, and optical sensors to analyze soil composition. The computing device 130 may send commands to configure the sensing device 120, such as adjusting the sampling rate, setting analysis parameters, or calibrating detection thresholds. The data acquisition process may allow for the identification of PFAS concentrations, soil moisture, organic carbon, and mineral composition.

In some embodiments, the computing device 130 may perform soil data analysis 530 to process sensor measurements and extract target contamination metrics. The analysis may be displayed at the user interface. The computing device 130 may execute preprocessing algorithms to filter noise from raw sensor data, normalize values across different measurement techniques, and compute key soil treatment parameters. The extracted data may include contamination severity, presence of PFAS precursors, and inferred degradation rates, which may inform subsequent optimization steps.

In some embodiments, in stage 540, the computing device 130 may control data acquisition and plant operations by sending treatment commands to the treatment device 110. The computing device 130 may regulate operational parameters such as milling speed, dwell time, chemical additive dosage, and energy input based on the analyzed soil properties. The computing device 130 may communicate with plant control systems using communication protocols such as CAN bus and analog interconnects.

In some embodiments, the computing device 130 may execute a process optimization algorithm 550 to determine ideal operational parameters dynamically. The optimization algorithm may incorporate rule-based logic, heuristic methods, and machine-learning models to refine treatment efficiency. Machine-learning models, such as Generalized Additive Models (GAMs) and Convolutional Neural Networks (CNNs), may predict treatment outcomes by analyzing hyperspectral imaging data and historical sensor measurements. The optimization process may balance energy use, chemical additive consumption, and reaction kinetics to ensure cost-effective and effective remediation.

In some embodiments, the computing device 130 may execute sensor data post-processing 560 to validate treatment performance and determine whether additional processing steps are required. The computing device 130 may compare pre-treatment and post-treatment contaminant concentrations to assess degradation efficacy. If PFAS concentrations exceed regulatory thresholds or fail to reach target destruction rates, the computing device 130 may update operational parameters for subsequent treatment cycles. The post-processing stage may also refine machine-learning models by incorporating new data to enhance predictive accuracy for future soil batches.

In some embodiments, the computing device 130 may store treatment and process data in a data store, such as the data store 140, for long-term analysis and process refinement. The data store 140 may take the form of a cloud database that may retain soil characterization data, real-time sensor measurements, treatment parameters, and optimization results. The computing device 130 may enable remote access to stored data for compliance reporting, performance validation, and historical trend analysis. The cloud-based architecture may facilitate multi-site treatment standardization.

In some embodiments, the data processing pipeline 500 may operate in a continuous feedback loop. The feedback loop may ensure treatment conditions are optimized dynamically based on real-time sensing, process monitoring, and historical data analysis. This closed-loop automation may reduce treatment costs, increase efficiency, and provide adaptable remediation solutions across diverse contamination scenarios.

### Optimization and Process Control

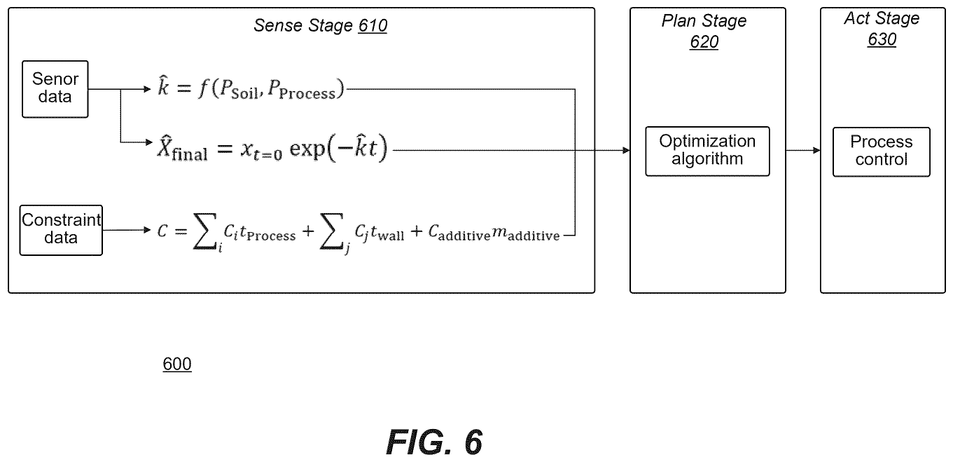

FIG. 6 is a conceptual diagram illustrating an example algorithm 600 that is modeled after a “Sense,” “Plan,” “Act” strategy, in accordance with some embodiments. The computing device 130 may receive sensor data, process the data using a machine-learning-based estimator, and execute an optimization routine that determines operational parameters for the treatment device 110. The computing device 130 may then transmit optimized process commands to the treatment device 110 to adjust treatment conditions dynamically.

In some embodiments, the computing device 130 may execute a sense stage 610 by applying a model to estimate a PFAS destruction rate constant based on soil and process parameters. The computing device 130 may utilize a Generalized Additive Model (GAM) to account for non-linear dependencies between various input factors, including soil composition, milling energy, and additive concentration. In some embodiments, the computing device 130 may apply a Convolutional Neural Network (CNN) to analyze hyperspectral imaging data obtained from near-infrared (NIR) spectrometry, identifying hidden contamination features that may not be detectable through conventional sensing methods. The computing device 130 may refine the model using data from a plurality of lab-scale experiments and dynamically update both input parameters and optimization variables as new data is collected. In some embodiments, the computing device 130 may model the final PFAS concentration using a first-order exponential decay function, capturing degradation kinetics in real time. Additionally, the computing device 130 may utilize coupled differential equation models to account for parameters such as crystal (silica) concentration and organic carbon levels. The computing device 130 may receive constraint data such as energy cost, additive cost, and other constraints that may or may not be related to costs. In some embodiments, the computing device 130 may incorporate a constraint function (e.g., a cost function) that evaluates trade-offs between energy consumption, equipment amortization, and chemical additive costs, ensuring that treatment conditions are optimized for both performance and efficiency.

In some embodiments, the computing device 130 may execute a plan stage 620 by utilizing the estimated PFAS destruction rate, the predicted final PFAS concentration, and the constraint function to determine optimal processing parameters. The computing device 130 may apply a Nelder-Mead constrained minimization method to iteratively refine operational parameters. The computing device 130 may optimize operational parameters until convergence across a range of input conditions. The computing device 130 may adjust process variables such as milling speed, dwell time, and additive concentration based on real-time sensing data and historical treatment performance.

In some embodiments, the computing device 130 may execute an act stage 630 by transmitting optimized control signals to the treatment device 110. The computing device 130 may interface with a process controller responsible for executing low-level commands that regulate treatment operations. In some embodiments, the process controller may receive updates to milling speed, chemical dosing, and energy input in response to real-time sensor feedback. The computing device 130 may initially apply this control strategy in a batched processing mode but may transition to a continuous process as system complexity and automation capabilities increase. By iteratively refining treatment parameters through the sense-plan-act strategy, the computing device 130 may enhance treatment efficiency, reduce processing time, and optimize resource utilization dynamically.

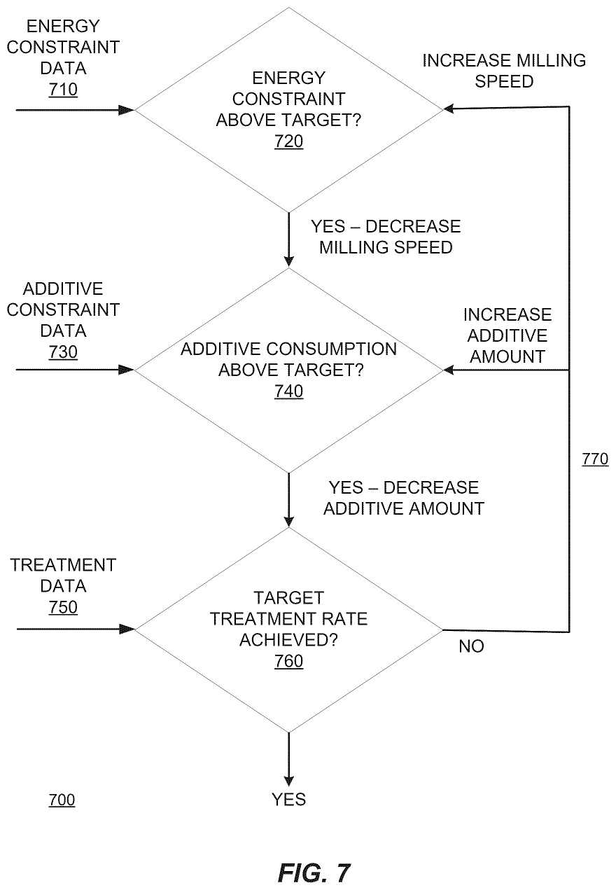

FIG. 7 is a flowchart depicting an example of an optimization algorithm 700, in accordance with some embodiments. The optimization algorithm 700 illustrated in FIG. 7 is merely an example of an optimization algorithm. In some embodiments, the computing device 130 may execute a closed-loop optimization process 700 for dynamically adjusting operational parameters during the treatment process of impacted soil. The computing device 130 may iteratively evaluate energy constraints, additive consumption, and treatment efficiency to optimize milling speed and chemical additive dosage in real time. The optimization algorithm 700 may be an example of optimization algorithm in the plan stage 620 in FIG. 6, but other optimization algorithms such as Nelder-Mead constrained minimization method, machine learning approach, may also be used in various embodiments.

In some embodiments, the computing device 130 may receive energy constraint data 710 as an input for determining whether the energy consumption associated with the treatment process is within a predefined target range. Constraint data may include cost data or any suitable constraint that is used to measure performance and/or efficiency of a treatment device 110. The computing device 130 may then determine whether the energy constraint exceeds a predefined threshold 720. If the energy consumption is above the target, the computing device 130 may decrease the milling speed to reduce energy usage. If the energy consumption is below the target, the computing device 130 may increase the milling speed to enhance treatment efficiency while maintaining energy cost-effectiveness.

In some embodiments, the computing device 130 may receive additive constraint data 730, which may represent the consumption levels of chemical additives used during the treatment process. The computing device 130 may determine whether additive consumption exceeds a predefined target 740. If additive consumption is above the target, the computing device 130 may decrease the additive amount to reduce excess usage and optimize material costs. If additive consumption is below the target, the computing device 130 may increase the additive amount to enhance the degradation of contaminants and improve overall treatment efficiency.

In some embodiments, the computing device 130 may receive treatment data 750, which may include level of contamination for a batch of impacted soil before the treatment, real-time analytical measurements of degradation levels, soil composition changes, and/or reaction kinetics. The computing device 130 may determine whether the target treatment rate has been achieved 760. If the target treatment rate has been met, the process may conclude, indicating successful remediation. If the target treatment rate has not been achieved, the computing device 130 may proceed to a feedback loop 770 to further refine treatment parameters.

In some embodiments, the computing device 130 may execute the feedback loop 770 by reevaluating energy consumption, additive usage, and treatment effectiveness in an iterative manner. The computing device 130 may reapply energy constraint data 710 and additive constraint data 730 to determine whether additional adjustments to milling speed and chemical additive dosing are required. If treatment performance remains below the target threshold, the computing device 130 may continuously refine operational parameters until optimal degradation efficiency is achieved. This feedback loop may ensure that soil batches with varying contamination levels receive treatment tailored to their specific characteristics, improving overall process adaptability and cost efficiency.

In some embodiments, the computing device 130 may execute this closed-loop control process iteratively, allowing dynamic adjustments to energy input and chemical additive use in response to real-time conditions. This process may enhance treatment efficiency, minimize unnecessary resource consumption, and ensure that PFAS degradation meets predetermined regulatory or performance thresholds.

### Example Machine Learning Models

In various embodiments, a wide variety of machine learning techniques may be used. Examples include different forms of supervised learning, unsupervised learning, and semi-supervised learning such as decision trees, support vector machines (SVMs), regression, Bayesian networks, and genetic algorithms. Deep learning techniques such as neural networks, including convolutional neural networks (CNN), recurrent neural networks (RNN), long short-term memory networks (LSTM), transformers, and linear recurrent neural networks such as Mamba may also be used. For example, various contaminant concentration estimations performed by a sensing module within the sensing device 120, parameter optimization calculations performed by the computing device 130, and other processes may apply one or more machine learning and deep learning techniques.

In various embodiments, the training techniques for a machine learning model may be supervised, semi-supervised, or unsupervised. In supervised learning, the machine learning models may be trained with a set of training samples that are labeled. For example, for a machine learning model trained to predict an optimal milling speed for PFAS degradation, the training samples may be historical treatment data that includes soil composition, initial PFAS concentrations, and energy inputs. The labels for each training sample may be binary or multi-class. In training a machine learning model for determining whether a soil batch requires pre-treatment, the training labels may include a positive label that indicates the presence of high moisture or interfering minerals requiring pretreatment adjustments and a negative label that indicates the soil is within standard processing parameters and does not require pretreatment. In some embodiments, the training labels may also be multi-class, such as different categories of PFAS degradation efficiency levels based on varied energy inputs and chemical additive dosages.

By way of example, the training set may include multiple past records of soil treatment batches with known outcomes. Each training sample in the training set may correspond to a previously treated batch of soil, and the corresponding outcome may serve as the label for the sample. A training sample may be represented as a feature vector that includes multiple dimensions. Each dimension may include data of a feature, which may be a quantized value of an attribute that describes the past record. For example, in a machine learning model that is used to optimize real-time treatment parameters for soil remediation, the features in a feature vector may include soil moisture content, organic carbon levels, initial PFAS concentration, mineral composition, milling speed, and additive concentration. In various embodiments, certain pre-processing techniques may be used to normalize the values in different dimensions of the feature vector.

In some embodiments, an unsupervised learning technique may be used. The training samples used for an unsupervised model may also be represented by feature vectors but may not be labeled. Various unsupervised learning techniques such as clustering may be used in determining similarities among the feature vectors, thereby categorizing the training samples into different clusters. In some cases, the training may be semi-supervised with a training set having a mix of labeled samples and unlabeled samples.

A machine learning model may be associated with an objective function, which generates a metric value that describes the objective goal of the training process. The training process may intend to reduce the error rate of the model in generating predictions. In such a case, the objective function may monitor the error rate of the machine learning model. In a model that generates predictions, the objective function of the machine learning algorithm may be the training error rate when the predictions are compared to the actual labels. Such an objective function may be called a loss function. Other forms of objective functions may also be used, particularly for unsupervised learning models whose error rates are not easily determined due to the lack of labels. In some embodiments, in determining optimal energy and additive input levels for soil treatment, the objective function may correspond to minimizing the overall treatment cost while ensuring target PFAS destruction levels are met within regulatory limits. In various embodiments, the error rate may be measured as cross-entropy loss, L1 loss (e.g., the sum of absolute differences between the predicted values and the actual value), L2 loss (e.g., the sum of squared distances).

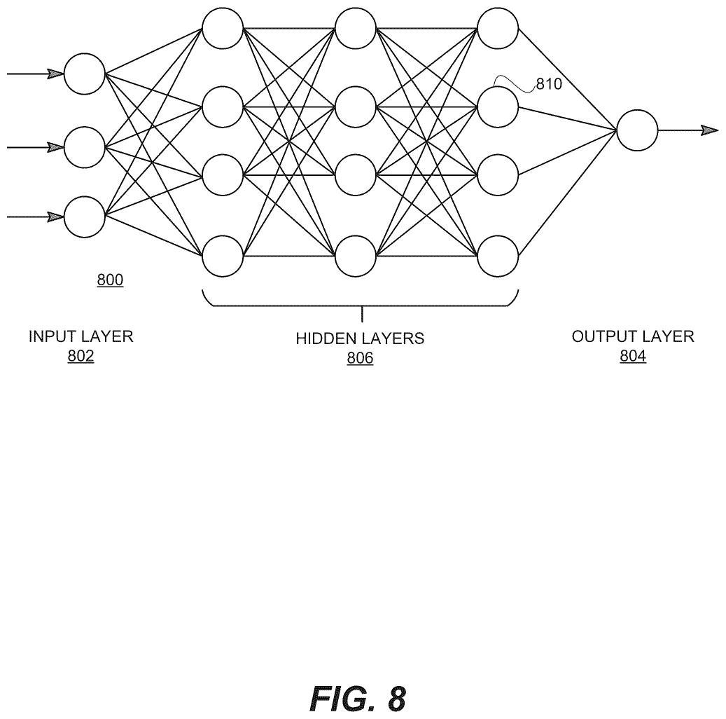

Referring to FIG. 8, a structure of an example neural network is illustrated, in accordance with some embodiments. The neural network 800 may receive an input and generate an output. The input may be the feature vector of a training sample in the training process and the feature vector of an actual case when the neural network is making an inference. The output may be the prediction, classification, or another determination performed by the neural network. The neural network 800 may include different kinds of layers, such as convolutional layers, pooling layers, recurrent layers, fully connected layers, and custom layers. A convolutional layer convolves the input of the layer (e.g., an image) with one or more kernels to generate different types of images that are filtered by the kernels to generate feature maps. Each convolution result may be associated with an activation function. A convolutional layer may be followed by a pooling layer that selects the maximum value (max pooling) or average value (average pooling) from the portion of the input covered by the kernel size. The pooling layer reduces the spatial size of the extracted features. In some embodiments, a pair of convolutional layers and pooling layers may be followed by a recurrent layer that includes one or more feedback loops. The feedback may be used to account for spatial relationships of the features in an image or temporal relationships of the objects in the image. The layers may be followed by multiple fully connected layers that have nodes connected to each other. The fully connected layers may be used for classification and object detection. In one embodiment, one or more custom layers may also be presented for the generation of a specific format of the output. For example, a custom layer may be used for estimating optimal treatment parameters based on inferred soil properties and predicted contaminant breakdown kinetics.

The order of layers and the number of layers of the neural network 800 may vary in different embodiments. In various embodiments, a neural network 800 includes one or more layers 802, 804, and 806, but may or may not include any pooling layer or recurrent layer. If a pooling layer is present, not all convolutional layers are always followed by a pooling layer. A recurrent layer may also be positioned differently at other locations of the CNN. For each convolutional layer, the sizes of kernels (e.g., 3×3, 5×5, 7×7, etc.) and the numbers of kernels allowed to be learned may be different from other convolutional layers.

Training of a machine learning model may include an iterative process that includes iterations of making determinations, monitoring the performance of the machine learning model using the objective function, and backpropagation to adjust the weights (e.g., weights, kernel values, coefficients) in various nodes 810. For example, a computing device 130 may receive a training set that includes historical soil treatment batches labeled with degradation efficiency and treatment cost. Each training sample in the training set may be assigned with labels indicating whether the treatment process achieved regulatory compliance with minimal resource use. The computing device 130, in a forward propagation, may use the machine learning model to generate predicted treatment efficiency scores. The computing device 130 may compare the predicted treatment efficiency scores with the labels of the training sample. The computing device 130 may adjust, in a backpropagation, the weights of the machine learning model based on the comparison. The computing device 130 backpropagates one or more error terms obtained from one or more loss functions to update a set of parameters of the machine learning model. The backpropagation may be performed through the machine learning model and one or more of the error terms based on a difference between a label in the training sample and the generated predicted value by the machine learning model.

The trained machine learning model can be used for performing real-time optimization of treatment parameters in a closed-loop soil remediation system or another suitable task for which the model is trained. In various embodiments, the training samples described above may be refined and used to re-train the model, improving the model's ability to perform inference tasks. This continuous training and re-training process may result in an adaptive system that enhances treatment efficiency through iterative learning cycles.

Multiple rounds of forward propagation and backpropagation may be performed. Training may be completed when the objective function has become sufficiently stable (e.g., the machine learning model has converged) or after a predetermined number of training iterations for a particular set of training samples. The trained machine learning model may then be deployed in the computing device 130 for real-time optimization of operational parameters in the closed-loop soil treatment system.

In various embodiments, the machine learning model may undergo a continuous retraining process to refine its accuracy and adaptability to different soil conditions. After the model is initially trained, multiple rounds of re-training may be performed based on updated treatment data. The computing device 130 may periodically retrain the machine learning model by obtaining additional sets of training data, such as new soil treatment records collected from the sensing device 120, changes in site-specific contamination patterns, and modifications to environmental regulations that define acceptable PFAS concentrations. The additional training data may also be generated through simulations or extrapolated from previous treatment results.

The computing device 130 may apply the additional training data to the machine learning model and adjust model parameters based on the updated dataset. In some embodiments, the retraining process may refine the model's ability to predict treatment effectiveness by incorporating new variables such as variability in PFAS precursor transformation, cost fluctuations in energy and additive consumption, or novel contamination patterns identified through hyperspectral imaging analysis. The additional training data may include any features and/or characteristics mentioned above, ensuring that the machine learning model remains responsive to evolving remediation needs.

In some embodiments, the machine learning model may be deployed within a distributed cloud-based architecture, allowing for seamless updates and integration of site-specific learning models. The computing device 130 may continuously upload real-time treatment data to a central data store 140, where optimization algorithms may refine global treatment strategies based on aggregated multi-site performance data. The cloud-based deployment may enable the computing device 130 to access optimized treatment models trained on a broader dataset, improving the adaptability of soil remediation strategies across different locations.

In various embodiments, the trained machine learning model may be integrated with decision-support systems to provide real-time recommendations to operators managing the remediation process. The computing device 130 may output optimized treatment parameters, including milling speed, dwell time, chemical additive dosage, and pre-treatment requirements, ensuring that each soil batch is processed under optimal conditions. Additionally, the computing device 130 may generate predictive alerts when real-time sensor data indicates a potential deviation from expected treatment performance, prompting corrective actions such as increasing milling energy, adjusting additive ratios, or extending processing time.

The closed-loop optimization enabled by the machine learning model may significantly improve treatment efficiency by reducing unnecessary energy consumption, minimizing chemical additive waste, and ensuring consistent regulatory compliance. The computing device 130 may use the continuously refined machine learning model to dynamically adapt treatment conditions, allowing for cost-effective and scalable soil remediation solutions across diverse contamination scenarios.

The integration of machine learning techniques within the computing device 130 may facilitate an adaptive, real-time optimization framework for soil treatment. By leveraging continuous feedback, historical data, and predictive modeling, the closed-loop remediation system may enhance process efficiency, reduce operational costs, and improve the effectiveness of PFAS degradation in contaminated soils.

### Computing Machine Architecture

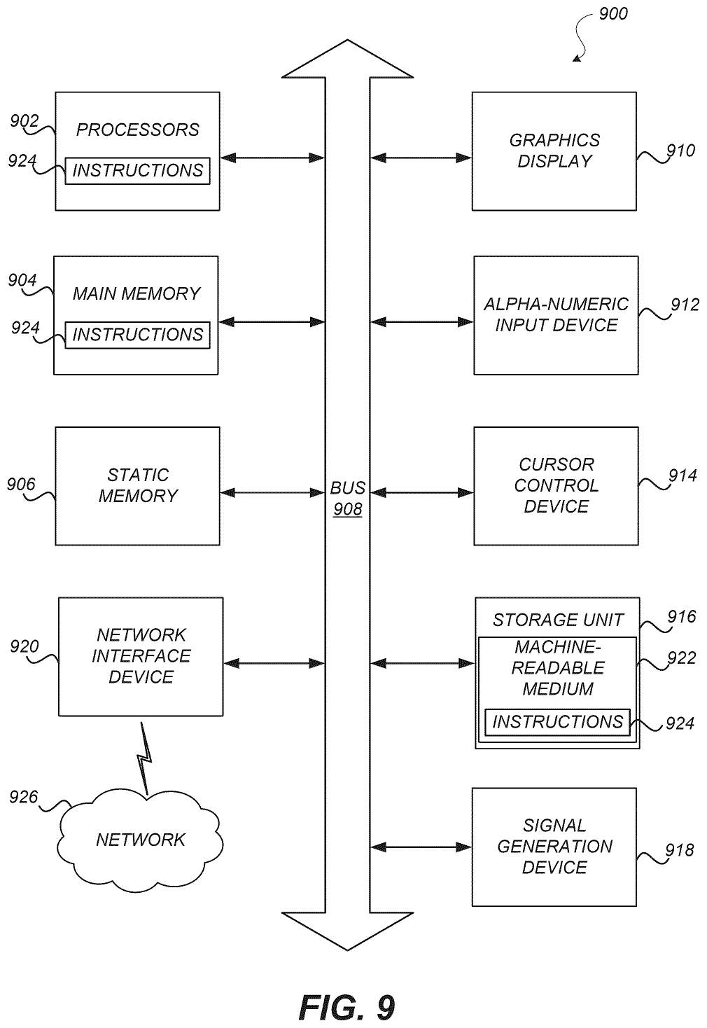

FIG. 9 is a block diagram illustrating components of an example computing machine that is capable of reading instructions from a computer-readable medium and executing them in a processor (or controller). A computer described herein may include a single computing machine shown in FIG. 9, a virtual machine, a distributed computing system that includes multiple nodes of computing machines shown in FIG. 9, or any other suitable arrangement of computing devices.

By way of example, FIG. 9 shows a diagrammatic representation of a computing machine in the example form of a computer system 900 within which instructions 924 (e.g., software, source code, program code, expanded code, object code, assembly code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 9 may correspond to some software, hardware, or combined components shown in FIGS. 1 and 2, including but not limited to, the sensing device 120 the computing device 130, the data store 140, the on-board compute 230. While FIG. 9 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 924 that specify actions to be taken by that machine. Further, while only a single machine is illustrated, the terms “machine” and “computer” may also be taken to include any collection of machines that individually or jointly execute instructions 924 to perform any one or more of the methodologies discussed herein.

The example computer system 900 includes one or more processors 902 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 900 may also include a memory 904 that stores computer code including instructions 924 that may cause the processors 902 to perform certain actions when the instructions are executed, directly or indirectly by the processors 902. Instructions can be any directions, commands, or orders that may be stored in different forms, such as equipment-readable instructions, programming instructions including source code, and other communication signals and orders. Instructions may be used in a general sense and are not limited to machine-readable codes. One or more steps in various processes described may be performed by passing through instructions to one or more multiply-accumulate (MAC) units of the processors.

One or more methods described herein improve the operation speed of the processor 902 and reduce the space required for the memory 904. For example, the database processing techniques and machine learning methods described herein reduce the complexity of the computation of the processors 902 by applying one or more novel techniques that simplify the steps in training, reaching convergence, and generating results of the processors 902. The algorithms described herein also reduce the size of the models and datasets to reduce the storage space requirement for memory 904.

The performance of certain operations may be distributed among more than one processor, not only residing within a single machine, but deployed across a number of machines. In some example embodiments, the one or more processors or processor-implemented modules may be located in a single geographic location (e.g., within a home environment, an office environment, or a server farm). In other example embodiments, one or more processors or processor-implemented modules may be distributed across a number of geographic locations. Even though the specification or the claims may refer to some processes to be performed by a processor, this may be construed to include a joint operation of multiple distributed processors. In some embodiments, a computer-readable medium comprises one or more computer-readable media that, individually, together, or distributedly, comprise instructions that, when executed by one or more processors, cause the one or more processors to perform, individually, together, or distributedly, the steps of the instructions stored on the one or more computer-readable media. Similarly, a processor comprises one or more processors or processing units that, individually, together, or distributedly, perform the steps of instructions stored on a computer-readable medium. In various embodiments, the discussion of one or more processors that carry out a process with multiple steps does not require any one of the processors to carry out all of the steps. For example, a processor A can carry out step A, a processor B can carry out step B using, for example, the result from the processor A, and a processor C can carry out step C, etc. The processors may work cooperatively in this type of situation such as in multiple processors of a system in a chip, in Cloud computing, or in distributed computing.

The computer system 900 may include a main memory 904, and a static memory 906, which are configured to communicate with each other via a bus 908. The computer system 900 may further include a graphics display unit 910 (e.g., a plasma display panel (PDP), a liquid crystal display (LCD), a projector, or a cathode ray tube (CRT)). The graphics display unit 910, controlled by the processor 902, displays a graphical user interface (GUI) to display one or more results and data generated by the processes described herein. The computer system 900 may also include an alphanumeric input device 912 (e.g., a keyboard), a cursor control device 914 (e.g., a mouse, a trackball, a joystick, a motion sensor, or other pointing instruments), a storage unit 916 (a hard drive, a solid-state drive, a hybrid drive, a memory disk, etc.), a signal generation device 918 (e.g., a speaker), and a network interface device 920, which also are configured to communicate via the bus 908.

The storage unit 916 includes a computer-readable medium 922 on which are stored instructions 924 embodying any one or more of the methodologies or functions described herein. The instructions 924 may also reside, completely or at least partially, within the main memory 904 or within the processor 902 (e.g., within a processor's cache memory) during execution thereof by the computer system 900, the main memory 904 and the processor 902 also constituting computer-readable media. The instructions 924 may be transmitted or received over a network 926 via the network interface device 920.

While computer-readable medium 922 is shown in an example embodiment to be a single medium, the term “computer-readable medium” should be taken to include a single medium or multiple media (e.g., a centralized or distributed database, or associated caches and servers) able to store instructions (e.g., instructions 924). The computer-readable medium may include any medium that is capable of storing instructions (e.g., instructions 924) for execution by the processors (e.g., processors 902) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### Additional Considerations

The foregoing description of the embodiments has been presented for the purpose of illustration; it is not intended to be exhaustive or to limit the patent rights to the precise forms disclosed. While particular embodiments and applications have been illustrated and described, it is to be understood that the invention is not limited to the precise construction and components disclosed herein and that various modifications, changes and variations which will be apparent to those skilled in the art may be made in the arrangement, operation and details of the method and apparatus disclosed herein without departing from the spirit and scope of the present disclosure. Persons skilled in the relevant art can appreciate that many modifications and variations are possible in light of the above disclosure.

The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

What is claimed is:

1. A system comprising:
a soil-treatment device for perfluoroalkyl and polyfluoroalkyl substances (PFAS) treatment, the soil-treatment device comprising:
an inlet for receiving PFAS-impacted soil from a PFAS-contaminated site, and
a reactor configured to carry out a mechanochemical operation to treat the PFAS-impacted soil according to a set of one or more operational parameters;
a sensing device that is on site with the soil treatment device, the sensing device configured to:
receive a sample from the PFAS-impacted soil, and
perform, on site during a treatment process of PFAS-impacted soil, a measurement of the sample; and
a computing device comprising memory and one or more processors, the memory storing executable instructions, wherein the executable instructions, when executed by the one or more processors, cause the one or more processors to:
receive the measurement of the sample performed by the sensing device;
apply a model to determine, during the treatment process of the PFAS-impacted soil and based on the measurement of the sample, a value of one of the operational parameters in the set; and
output the value for the soil treatment device to carry out the mechanochemical operation according to the set of one or more operational parameters.

2. The system of claim 1, wherein the reactor comprises a ball mill reactor that comprises a rotary shaft and metallic ball bearings, the mechanochemical operation comprises stirring the PFAS-impacted soil with the metallic ball bearings at a rotational speed, and the rotational speed is adjustable and is one of the operational parameters in the set.

3. The system of claim 1, wherein the reactor is configured to operate at a temperature that is below 50 degrees Celsius and below 1.5 times atmospheric pressure.

4. The system of claim 1, wherein the reactor is configured to receive aluminum and magnesium metallic additives for the mechanochemical operation, and an amount of the metallic additive is one of the operational parameters.

5. The system of claim 1, wherein the sensing device is an automatic sensor that is installed within the soil-treatment device.

6. The system of claim 1, wherein the sensing device include one or more of:
a direct ionization mass spectrometer, a near-infrared (NIR) spectroscopy sensor, thermal sensor, or optical and spectral sensors.

7. The system of claim 1, wherein the sensing device is a measurement device separated from the soil-treatment device, and the sensing device comprises laboratory kits to measure the sample during the treatment process.

8. The system of claim 1, wherein the treatment process comprises a pre-operation measurement and the mechanochemical operation.

9. The system of claim 1, wherein the set of one or more operational parameters includes one or more of:
a dwell time, a milling speed, a chemical additive amount, or an energy input.

10. The system of claim 1, wherein the model is a generalized additive model and the generalized additive model is configured to predict adjustments to operational parameters based on one or more sensed soil parameters.

11. The system of claim 1, wherein the model is a convolutional neural network and the convolutional neural network is configured to analyze hyperspectral imaging data from Near-Infrared (NIR) spectroscopy to determine hidden features in soil composition and contamination patterns.

12. The system of claim 1, wherein the measurement performed by the sensing device includes one or more of:
a contaminant concentration, a moisture content, an organic carbon measurement, and a mineral composition.

13. The system of claim 1, wherein the model comprises algorithms for:
measuring soil parameters;
estimating operational parameters based on the soil parameters;
receiving energy cost data; and
adjusting the operational parameters based on the energy cost data.

14. The system of claim 1, wherein the executable instructions, when executed, further cause the one or more processors to:
determine an estimated operational parameter based on soil parameters;
calculate the estimated destruction rate;
identify a target treatment end point;
provide the estimated destruction rate, the target treatment end point, and the estimated operational parameter into an optimization algorithm to adjust the operational parameter based on cost; and
output a recommended action that includes one or more of: a pre-treatment step, a post-treatment step, or a value of the operational parameter.

15. The system of claim 1, wherein the PFAS-impacted soil is a first batch of PFAS-impacted soil from a contaminated site, the contaminated site comprises the first batch and a second batch of PFAS-impacted soil, and wherein the second batch of PFAS-impacted soil has a level of contamination that is at least 10 times different from the first batch, and the computing device is configured to determine batch-specific operational parameters for treating the batches.

16. The system of claim 1, wherein the model is configured to determine the value of one of the operational parameters based on energy cost.

17. A method for treating soil with perfluoroalkyl and polyfluoroalkyl substances (PFAS) contamination, the method comprising:
causing an excavation of PFAS-impacted soil from a PFAS-contaminated site;
performing, on site during a treatment process of the PFAS-impacted soil, a measurement of the sample;
applying a model to determine, during the treatment process of the PFAS-impacted soil and based on the measurement of the sample, a value of an operational parameter for operating a soil-treatment device that carries out the treatment process that includes a mechanochemical operation to treat the PFAS-impacted soil according to the operational parameter; and
updating the operational parameter during the treatment process based on live data of further measurements of samples from the PFAS-impacted soil.

18. The method of claim 17, wherein the set of one or more operational parameters includes one or more of:
a dwell time, a milling speed, a chemical additive amount, or an energy input.

19. The method of claim 17, wherein the model is a generalized additive model and the generalized additive model is configured to predict adjustments to operational parameters based on one or more sensed soil parameters.

20. The method of claim 17, wherein the model is a convolutional neural network and the convolutional neural network is configured to analyze hyperspectral imaging data from Near-Infrared (NIR) spectroscopy to determine hidden features in soil composition and contamination patterns.
